(19) United States
(12) Patent Application Publication
(10) Pub. No.: US 2016/0128729 A1
(43) Pub. Date: May 12, 2016

(54) DEVICES AND METHODS FOR MANIPULATING BODILY TISSUE

(71) Applicant: BIOCEPTIVE, INC., New Orleans, LA (US)

(72) Inventors: Shuchi P. KHURANA, Metairie, LA (US); Benjamin D. CAPPIELLO, New Orleans, LA (US); Alan BACHMAN, Milford, CT (US); Jeffrey RANSDEN, Fairfield, CT (US)

(73) Assignee: Bioceptive, Inc., New Orleans, LA (US)

(21) Appl. No.: 14/897,367

(22) PCT Filed: Jun. 20, 2014
(86) PCT No.: PCT/US14/43417

§ 371 Date: Dec. 10, 2015

Related U.S. Application Data
(60) Provisional application No. 61/837,497, filed on Jun. 20, 2013.

(51) Int. Cl. A61B 17/42; A61F 6/18

(52) U.S. Cl. CPC A61B 17/4241; A61F 6/18; A61B 2017/00566

(57) ABSTRACT

A device includes an insertion member having a distal end portion configured to be removably engaged with an implant, and a sheath having an exit portion and defining a lumen. The exit portion of the sheath includes a set of dilation members configured to be moved from a first configuration to a second configuration. The set of dilation members forms a dilation surface when the set of dilation members is in the first configuration and defines an opening when the set of dilation members is in the second configuration. The sheath includes a hinge configured to facilitate movement of the set of dilation members between the first configuration and the second configuration. The distal end portion of the insertion member configured to move within the lumen to convey the implant from within the lumen via the opening when the set of dilation members is in the second configuration.

### CROSS-REFERENCE TO RELATED APPLICATIONS

This application claims priority to and the benefit of U.S. Provisional Patent Ser. No. 61/837,497 entitled, “Device and Methods for Manipulating Bodily Tissue,” filed Jun. 20, 2013, the disclosure of which is incorporated herein by reference in its entirety.

The embodiments described herein relate generally to devices and methods for manipulating bodily tissue. More particularly, the embodiments described herein relate to devices and methods for inserting an implant into a body cavity and attaching and pulling traction of a target tissue such as, the cervix.

Difficulty of insertion is a hurdle to the more widespread use of known intrauterine devices (IUDs) by physicians and health care workers worldwide. One disadvantage of known methods for IUD insertion relate to the multi-step nature of such known methods. In particular, known methods of inserting the IUD involve up to five separate medical instruments in addition to a vaginal speculum, namely: a cervical tenaculum, a uterine sound, an Os finder (when needed), an IUD inserter, and surgical scissors (to trim IUD strings to length).

The cervical tenaculum is used in many intrauterine procedures. This includes, though is not limited to, artificial insemination (intrauterine semination), colcoscopy, dilation and curettage, manual vacuum aspiration, electric vacuum aspiration, endometrial biopsy, dilatation and evacuation, insertion of various contraceptive devices, and certain abortion procedures. The tenaculum is a crude device consisting of a scissor-like handle with two sharp prongs that pierce the tissue of a woman's cervix when attachment to the cervix is made, which can cause undue pain and/or damage to the cervical tissue.

Additionally, some methods for inserting an IUD include dilating the cervix using a cervical dilator or os finder, adding an additional step to insertion procedure. This dilation allows the IUD device deployment tube to enter the cervix and implant the device. Such dilations are performed for many similar procedures involving the uterus, such as colcoscopy, dilation and curettage, manual vacuum aspiration, electric vacuum aspiration, endometrial biopsy, dilation and evacuation, gynecological brachy therapy, insertion of various contraceptive devices, and certain abortion procedures. These dilations are performed to prevent damage to the tissue during insertion. For example, damage to the tissue can be caused by the physical act of insertion as the distal tip of the insertion member can scrape or catch on surrounding cervical tissue. Furthermore, the deployment tube of known insertion devices can exert excessive pressure while detecting tissue at the distal tip, which can result in trauma at the detected tissue site. In some cases, the health care provider may choose to forego the use of a cervical dilator in belief that the uterine sound can perform, the similar function of dilating the cervical canal and also creating an established passageway through which the IUD inserter will enter, however, this can be a dangerous part of IUD insertion during which many perforations (creation of false passageways) can occur.

Thus, a need exists for improved devices and methods for attachment to and manipulation of bodily tissue, for example, the cervix, to facilitate an intrauterine procedure.

### SUMMARY

Devices and methods for inserting an implant and/or drug into a bodily cavity such as, for example, the cervix, axe described herein. In some embodiments, a device includes an insertion member and a sheath. The insertion member has a distal end portion configured to be removably engaged with an implant. The sheath has an exit portion and defines a lumen. The exit portion of the sheath includes a set of dilation members configured to be moved from a first configuration to a second configuration. The set of dilation members forms a dilation surface when the set of dilation members is in the first configuration. The set of dilation members defines an opening when the set of dilation members is in the second configuration. The sheath includes a hinge configured to facilitate movement of the set of dilation members between the first configuration and the second configuration. The distal end portion of the insertion member is configured to move within the lumen to convey the implant from within the lumen via the opening when the set of dilation members is in the second configuration.

Devices and methods for attaching and applying traction on a target tissue to facilitate the insertion of an instrument, implant and/or drug into a body cavity are also described herein. In some embodiments, a device includes a connection portion and an engagement portion. The connection portion is configured to be pivotably coupled to a delivery device and includes a vacuum port configured to be coupled to a vacuum source. The engagement portion is coupled to the connection portion and includes a rib and an inner surface. The inner surface defines at least a portion of a vacuum pathway and at least a portion of a suction volume. The suction volume is in fluid communication with the vacuum port via the vacuum pathway and is configured to receive a first portion of a target tissue when a portion of the rib is engaged with the target tissue and a vacuum is applied to the vacuum port. The inner surface is configured such that the vacuum pathway provides continuous communication between the vacuum port and the suction volume when the first portion of the target tissue is within the suction volume. The rib is configured to be in contact with a second portion of the target tissue when the first portion of target tissue is disposed in the suction volume to limit movement of the target tissue out of the suction volume.

### BRIEF DESCRIPTION OF THE DRAWINGS

FIG. 1 is an illustration of a portion of the female reproductive system provided for reference.

FIGS. 2 and 3 are schematic illustrations of a portion of a delivery device according to an embodiment, in a first configuration and a second configuration, respectively.

FIGS. 4 and 5 are a top view and a side view, respectively, of a portion of a sheath according to an embodiment, in a first configuration.

FIG. 6 is a side view of the portion of the sheath of FIG. 4 in a second configuration.

FIG. 7 is a schematic illustration of a portion of a tissue engagement device according to an embodiment, disposed about a portion of a target tissue.

FIGS. 8 and 9 are a front perspective view and a rear perspective view, respectively, of a portion of a medical device according to an embodiment.

FIGS. 10 and 11 and a front perspective view and a rear perspective view of a vacuum head included in the delivery device of FIG. 8.

FIG. 12 is a cross-sectional view of the vacuum head of FIG. 10 taken along the line 12-12.

FIG. 13 is the cross-sectional view of FIG. 12 illustrating the vacuum head in contact with a portion of the uterus.

FIG. 14 is the cross-sectional view of a vacuum head according to an embodiment.

FIGS. 15 and 16 are a front perspective view and a rear perspective view of a vacuum head according to an embodiment.

FIG. 17 is the cross-sectional view of the vacuum head of FIG. 15 taken along the line 17-17.

FIGS. 18 and 19 are a front perspective view and a rear perspective view of a vacuum head according to an embodiment.

FIG. 20 is the cross-sectional view of the vacuum head of FIG. 18 taken along the line 20-20.

FIG. 21 is a side view of the vacuum head of FIG. 18.

FIGS. 22-24 are cross-sectional view of the vacuum head of FIG. 18 taken along the lines 22-22, 23-23, and 24-24 in FIG. 21, respectively.

FIGS. 25 and 26 are a front perspective view and a rear perspective view of a vacuum head according to an embodiment.

FIG. 27 is a side view of the vacuum head of FIG. 25.

FIGS. 28 and 29 are cross-sectional view of the vacuum head of FIG. 25 taken along the lines 28-28 and 29-29 in FIG. 27, respectively.

FIGS. 30 and 31 are a perspective view and a side view, respectively, of a portion of a sheath according to an embodiment.

FIG. 32 is a front view of the sheath of FIG. 30 in a first configuration.

FIGS. 33 and 34 are a perspective view and a side view, respectively, of a portion of a sheath according to an embodiment.

FIGS. 35 and 36 are a perspective view and a side view, respectively, of a portion of a sheath according to an embodiment.

FIGS. 37 and 38 are a perspective view and a front view, respectively, of a portion of a sheath according to an embodiment.

### DETAILED DESCRIPTION

In some embodiments, a delivery device and/or tissue manipulation device of that the types described herein can facilitate an intrauterine procedure. The embodiments described herein can reduce the risk of complications due to poor insertion technique and can increase the ease of insertion of, for example, an intrauterine device (IUD). The devices shown and described herein can also be used to insert any another device, implant and/or pharmaceutical into a female reproductive system. In some embodiments, the devices and methods described herein can be used for insertion of a catheter, enema, drug delivery object, imaging tools, endoscopy, tubes (e.g., into the lungs and other body cavities), or other applications where precise insertion would be beneficial to the efficacy of the treatment and/or to eliminate complications or pain. Furthermore, the devices and methods described herein can provide gentler and/or easier approaches for navigating around and/or past obstacles or anatomical variations in bodily passageways, while also preventing trauma from excess pressure when detecting tissues with the distal tip of the insertion member.

In some embodiments, any of the devices described herein can be a disposable and/or comprehensive device that can, inter alia, facilitate insertion of an IUD to a desired and/or predetermined position and/or orientation within the body. The embodiments described herein can improve known procedures that employ up to five separate medical instruments by allowing substantially the same procedures to be completed using a single device (e.g., any of the devices described herein). By so doing, the embodiments described herein can make a procedure of inserting an IUD more intuitive and easier to perform, thereby decreasing the amount of adverse events, mainly accidental expulsions, while also expanding access to IUDs worldwide by providing a delivery device that one can operate with minimal training. The embodiments described herein are configured to reduce or eliminate perforation of the tissue of the cervix (e.g., resulting from the use of a cervical tenaculum) and uterus by including mechanisms that limit forces applied during the insertion process. The embodiments described herein can also increase the probability of placing an IUD as close to the fundus of the uterus as possible compared to the placement of an IUD using other devices and/or methods.

In some embodiments, a delivery device can be configured to articulate with the cervix and can be used, for example, to insert an IUD into a woman's uterus with no other tools needed, and without the need for exceptional skill and/or training. Moreover, the embodiments described herein can increase, for example, ease of use, repeatability, and precision of insertion. Thus, after a short training session, a health care practitioner can properly insert an IUD safely using the devices and according to the methods described herein. Moreover, some embodiments described herein can be used with additional tools that are currently used in IUD insertions and/or other procedures.

The embodiments described herein need not be limited to use for inserting IUDs, and can also be used in connection with any suitable procedure. Moreover, certain embodiments, such as the suction heads and/or tissue engagement devices described herein can be used independently from other embodiments described herein. For example, the tissue engagement devices (e.g., the device 400 described below) can be used without the sheaths and/or insertion devices described herein (e.g., the sheath 260). For example, in some embodiments, the medical device 400 can be used to engage, manipulate and/or position a portion of the anatomy to facilitate any suitable procedure.

In some embodiments, a device includes an insertion member and a sheath. The insertion member has a distal end portion configured to be removably engaged with an implant. The sheath has an exit portion and defines a lumen. The exit portion of the sheath includes a set of dilation members configured to be moved from a first configuration to a second configuration. The set of dilation members forms a dilation surface when the set of dilation members is its the first configuration. The set of dilation members defines an opening when the set of dilation members is in the second configuration. The sheath includes a hinge configured to facilitate movement of the set of dilation members between the first configuration and the second configuration. The distal end portion of the insertion member is configured to move within the lumen to convey the implant from within the lumen via the opening when the set of dilation members is in the second configuration.

In some embodiments, a device includes an insertion member and a sheath. The insertion member has a distal end portion configured to be removably engaged with an implant to move the implant in a distal direction. The sheath has a distal end portion, an exit portion and defines a lumen. The lumen is configured to receive at least a portion of the insertion member and the implant. The distal end portion has a continuous dilation surface spaced apart from the lumen. The exit portion defines an opening in communication with the lumen, and includes an exit surface defining an end portion of the lumen. The exit surface is configured to contact a distal end portion of the implant when the insertion member moves the implant in the distal direction relative to the sheath to convey the implant from within the lumen via the opening.

In some embodiments, a delivery device includes a connection portion and an engagement portion. The connection portion is configured to be pivotably coupled to a delivery device and includes a vacuum port configured to be coupled to a vacuum source. The engagement portion is coupled to the connection portion and includes a rib and an inner surface. The inner surface defines at least a portion of a vacuum pathway and at least a portion of a suction volume. The suction volume is in fluid communication with the vacuum port via the vacuum pathway and is configured to receive a first portion of a target tissue when a portion of the rib is engaged with the target tissue and a vacuum is applied to the vacuum port. The inner surface is configured such that the vacuum pathway provides continuous communication between the vacuum port and the suction volume when the first portion of the target tissue is within the suction volume. The rib is configured to be in contact with a second portion of the target tissue when the first portion of target tissue is disposed in the suction volume to limit a movement of the target tissue out of the suction volume.

In some embodiments, a device includes a suction mechanism configured to articulate, at least partially, with a target tissue when the device is inserted into a body cavity. The suction mechanism includes an elongate portion and an inner volume. The elongate portion extends from the suction mechanism to be inserted into a portion of the body cavity. The inner volume is fluidically coupled to a vacuum source.

As used in this specification, the singular forms “a,” “an” and “the” include plural reference unless the context clearly dictates otherwise. Thus, for example, the term “a member” is intended to mean a single member or a combination of members, “a material” is intended to mean one or more materials, or a combination thereof.

As used herein, the words “proximal” and “distal” refer to direction closer to and away from, respectively, an operator of the medical device. Thus, for example, the end of the medicament delivery device contacting the patient's body would be the distal end of the medicament delivery device, while the end opposite the distal end would be the proximal end of the medicament delivery device.

As used herein, the term “stiffness” is related to an object's resistance to deflection, deformation, and/or displacement that is produced by an applied force, and is generally understood to be the opposite of the object's “flexibility.” For example, a wall with greater stiffness is more resistant to deflection, deformation and/or displacement when exposed to a force than a wall having a lower stiffness. Similarly stated, an object having a higher stiffness can be characterized as being more rigid than an object having a lower stiffness. Stiffness can be characterized in terms of the amount of force applied to the object and the resulting distance through which a first portion of the object deflects, deforms, and/or displaces with respect to a second portion of the object. When characterizing the stiffness of an object, the deflected distance may be measured as the deflection of a portion of the object different irons the portion of the object to which the force is directly applied. Said another way, in some objects, the point of deflection is distinct from the point where force is applied.

Stiffness (and therefore, flexibility) is an extensive property of the object being described, and thus is dependent upon the material from which the object is formed as well as certain physical characteristics of the object (e.g., cross-sectional shape, length, boundary conditions, etc.). For example, an object having a length and a cross-sectional area may have a greater stiffness than an object having an identical length but a smaller cross-sectional area. In some instances, however, the nature and/or use of the object can, inter alia, limit a range of sizes and/or cross-sectional areas and thus, the stiffness of the object cannot be increased or decreased by changing some physical characteristics of the object. As such, in some instances, the stiffness of an object can be increased or decreased by selectively including in the object a material having a desired modulus of elasticity, flexural modulus, and/or hardness. The modulus of elasticity is an intensive property of (i.e., is intrinsic to) the constituent material and describes an object's tendency to elastically (i.e., non-permanently) deform in response to an applied force. A material having a high modulus of elasticity will not deflect as much as a material having a low modulus of elasticity in the presence of an equally applied stress. Thus, the stiffness of the object can be decreased, for example, by introducing into the object and/or constructing the object of a material having a relatively low modulus of elasticity.

In another example, the stiffness of the object can be increased or decreased by changing the flexural modulus (also an intensive property) of a material from which the object is constructed. Flexural modulus is used to describe the ratio of the applied stress on an object in flexure to the corresponding strain in the outermost portions of the object. The flexural modulus, rather than the modulus of elasticity, is used to characterize certain materials, for example plastics, that do not have material properties that are substantially linear over a range of conditions. An object with a first flexural modulus is less elastic and has a greater strain on the outermost portions of the object than an object with a second flexural modulus lower than tire first flexural modulus. Thus, the stiffness of an object can be increased by including in the object a material having a high flexural modulus.

Similarly, a material's hardness is an intensive property of the constituent material and describes the measure of how resistant the material is to various kinds of permanent shape change (i.e., plastic deformation) when a force is applied. In discussing the hardness and the subsequent effect on the stiffness of an object, the Shore durometer scale is often used. There are several scales for Shore durometers with two commonly used in describing plastics, polymers, elastomers, and/or rubbers, namely, type A and type D, where type A is generally used for softer materials and type D is generally used for harder materials. The Shore durometer of a material is denoted by a number between 0 and 100, with higher numbers indicating a harder material, followed by the type of scale. For instance, a first material can be measured as having a Shore durometer of 40 Shore A and a second material can be measured as having a Shore durometer of 60 Shore D. Therefore, according to the Shore durometer scale, the second material is harder and thus, more stiff than the first material.

The embodiments described herein can be formed or constructed of one or more biocompatible materials. Examples of suitable biocompatible materials include metals, glasses, ceramics, or polymers. Examples of suitable metals include pharmaceutical grade stainless steel, gold, titanium, nickel, iron, platinum, tin, chromium, copper, and alloys thereof. A suitable polymer may be biodegradable or non-biodegradable. Examples of suitable biodegradable polymers include polylactides, polyglycolides, polylactide-coglycolides (PLGA), polyanhydrides, polyorthoesters, polyetheresters, polycaprolactones, polyesteramides, poly(butyric acid), poly(valeric acid), polyurethanes and copolymers and blends thereon. Examples of suitable non-biodegradable polymers include nylons, polyesters, polycarbonates, polyacrylates, polymers of ethylene-vinyl acetates and other acyl-substituted cellulose acetates, non-degradable polyurethanes, polystyrenes, polyvinyl chloride, polyvinyl fluoride, polyvinyl imidazole), chlorosulphonate polyolefins, polyethylene oxide, blends and copolymers thereof. Moreover, the embodiments described herein can be formed or constructed of one or more of the biocompatible materials and/or blends thereof based at least in part of a durometer of the constituent biocompatible material.

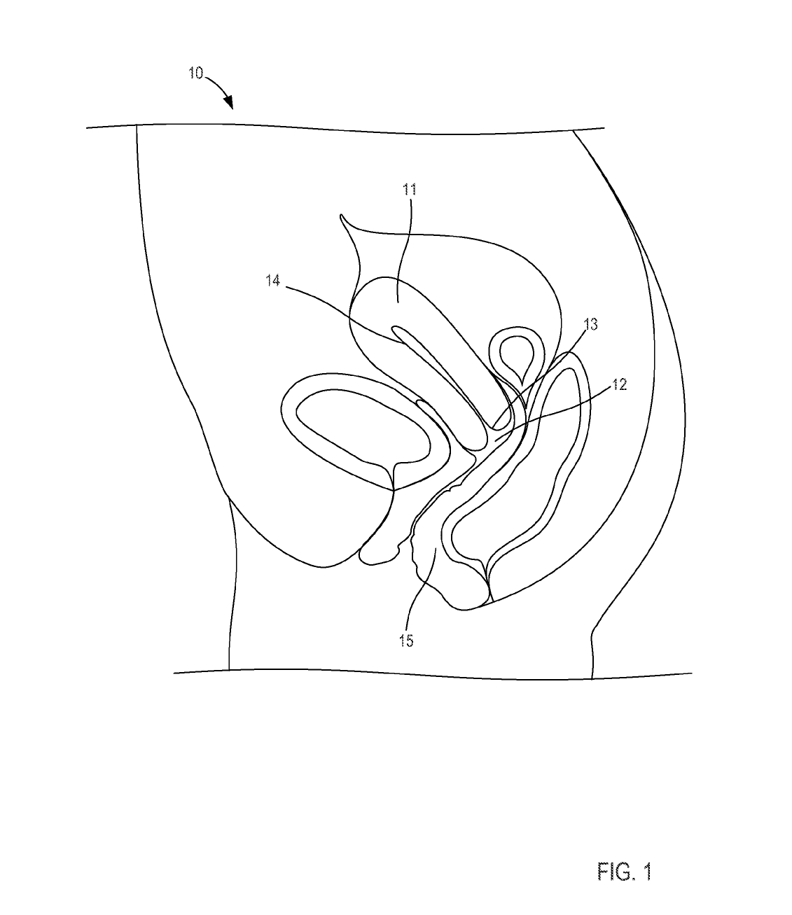

FIG. 1 is an illustration of a portion of the female reproductive system shown, for example, to provide context to the description of the devices and methods herein. That is to say, while specific portions of the female reproductive system are shown and described, it is not meant to be an exhaustive discussion of the female reproductive system. Rather, pertinent anatomical structures, passageways, etc. are presented by way of example to illustrate a use of the devices and methods described herein. While the female reproductive system is shown and described in FIG. 1, the devices and methods described herein can be used in other portions of the human body (e.g., either male or female). As shown in FIG. 1, a pelvic region of the female body 10 (also referred to as “body”) includes, inter alia, the vagina 15 and the uterus 11. More particularly, the uterus 11 is a substantially U-shaped or pearl-shaped and is positioned immediately posterior to the urinary bladder and in communication with the vaginal canal. The uterus 11 includes a neck portion known as the cervix 12, which defines a cervical os 13 providing access to an interior region of the uterus. Opposite the cervical os 13 (also referred to herein as “os”) is a portion of the uterus 11 known as the fundus 14. In some intrauterine procedures such as, for example, the insertion of an intrauterine device (IUD), it is desirable to deliver an implant through the vagina 15 and the os 13 to be implanted into a portion of the fundus 14. As is shown in FIG. 1, the insertion path is generally tortuous and often manipulation of a portion of the cervix 12 is used to allow access through the os 13, as described in further detail herein.

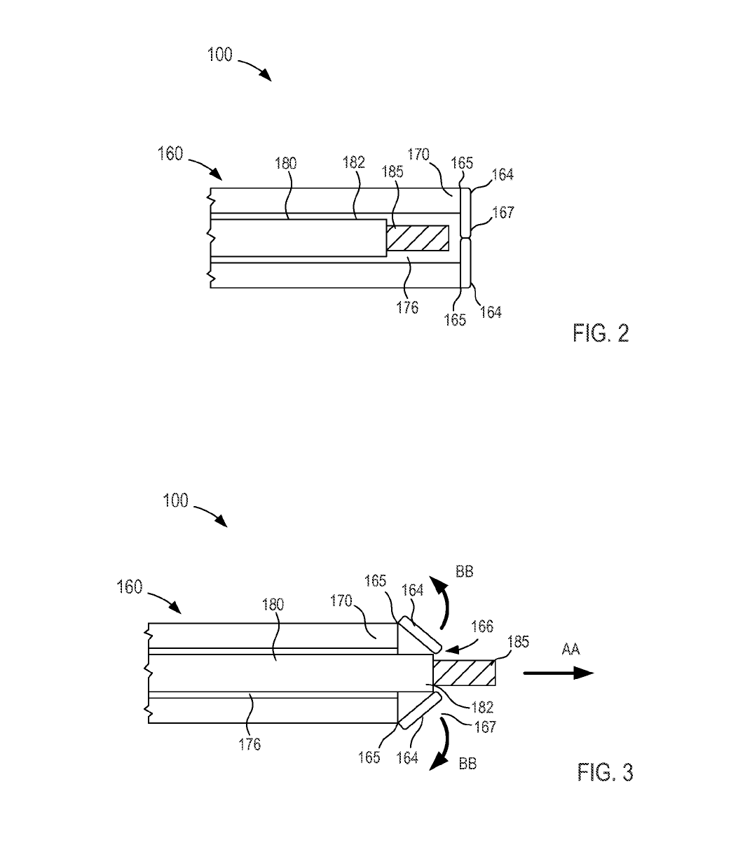

FIGS. 2 and 3 are schematic illustrations of a portion of a delivery device 100 according to an embodiment. Such a delivery device 100 can be used, for example, to deliver an implant 185 to a target location within the body. For example, in some embodiments, such a delivery device can be used to place an intrauterine device (IUD) in contact with the fundus 14 of or otherwise within the uterus 11 (see e.g., FIG. 1). The portion of the delivery device includes a sheath 160 that can be coupled to any suitable portion of a delivery device. In some embodiments, the sheath 160 can be movably coupled to and/or movably disposed within a portion of a handle or the like of a delivery device (not shown in FIGS. 2 and 3). Such a delivery device can include, for example, an actuator or the like that can be operable in moving the sheath 160 relative to the handle. For example, in some embodiments, the sheath 160 can be included in a delivery device similar to those described in U.S. Patent Application Publication No. 2013/0291872 entitled, “Methods and Apparatus for Inserting a Device or Pharmaceutical Into a Body Cavity,” filed on Apr. 16, 2013 as U.S. application Ser. No. 13/863,734 ('734 application) and/or PCT Publication No. WO2013/082452 entitled, “Methods and Apparatus for Inserting a Device or Pharmaceutical Into a Uterus,” filed on Nov. 30, 2012 as PCT Application No. PCT/U.S.2012/067335 ('335 application), the disclosures of which are incorporated herein by reference in their entireties. In some embodiments, such an insertion device can include, for example a vacuum head (also referred to as a vacuum nozzle) or the like that can be used to engage a portion of the cervix 12 and once engaged, a user can pull traction and/or can otherwise manipulate the cervix 12 to facilitate the insertion of the sheath 160 through the cervical os 13.

The sheath 160 can be formed from any suitable material or combination of materials such as, for example, those described above. More specifically, the sheath 160 can be formed or constructed from a substantially flexible material (e.g., a relatively high durometer rubber, siliconized rubber, polypropylene, polyethylene, and/or the like) that can allow for bending, twisting, opening, and/or otherwise reconfiguring of at least a portion of the sheath 160. For example, the sheath 160 can be sufficiently flexible to be advanced along a tortuous path defined by a portion of the body, yet can be sufficiently stiff to resist kinking, buckling, collapsing, and/or plastically deforming. In some embodiments, the sheath 160 can have any suitable hardness. For example, in some embodiments, the sheath 160 can have a Shore durometer between about 60 Shore D and about 90 Shore D.

As shown, the sheath 160 includes at least an exit portion 170 and defines a lumen 176. The lumen 176 movably receives a portion of an insertion member 180 having a distal end portion 182 that is configured to be placed in contact with the implant 185. For example, the implant 185 can be loaded into the sheath 160 to be movably disposed in the lumen 176 and similarly, the insertion member 180 can be inserted into the lumen 176 to be placed in contact with the implant 185. In some embodiments, the exit portion 170 can be a distal end portion of the sheath 160 and the lumen 176 can be configured to extend therethrough. In other embodiments, the exit portion 170 can be spaced apart from a distal surface of the sheath 160, as described in further detail herein.

The exit portion 170 of the sheath 160 includes or is otherwise coupled to a set of dilation members 164. For example, in some embodiments, the set of dilation members 164 can be monolithically formed with the sheath 160 (i.e., the exit portion 170 of the sheath 160). In other embodiments, the set of dilation members 164 can be included in, for example, a distal tip or the like that is constructed separately from and can be coupled to the exit portion 170 of the sheath 160. For example, in some embodiments, such a distal tip can be an over-mold or the like. In other embodiments, such a distal tip can be formed from a material that is co-extruded with the sheath 160. In such embodiments, the distal tip and the set of dilation members 164 included therein can be formed from a substantially flexible material with a relatively low hardness (e.g., different from the material forming the sheath 160). For example, such a distal tip and/or the dilation members 164 can be formed from a relatively low durometer rubber, silicone, siliconized rubber, and/or the like. As such, the set of dilation members 164 and/or the distal tip can have a hardness that is less than a hardness of the other portions of the sheath 160. For example, the distal tip can be formed from a material having a Shore durometer between about 55 Shore A and about 75 Shore A. In some embodiments, the distal tip can be formed from a substantially fluid-impermeable foam, such as foam rubber or the like. In this manner, the relatively low hardness of the distal tip can, for example, limit and/or substantially prevent damage to bodily tissue as the sheath 160 is inserted into the body.

As shown in FIGS. 2 and 3, the dilation members 164 can be included in or otherwise coupled to the exit portion 170 in such a manner as to allow the dilation members 164 to be moved and/or transitioned between a first configuration (FIG. 2) and a second configuration (FIG. 3). For example, as shown, each dilation member 164 is movably coupled to the exit portion 170 of the sheath 160 via a hinge 165 or the like. Each hinge 165 can be, for example, a living hinge or the like that can be configured to deform in a predetermined manner to move the corresponding dilation member 164 relative to the sheath 160. More specifically, dilation members 164 can be monolithically formed with the exit portion 170 of the sheath 160 and the exit portion 170 can include a surface that is thinned, stretched, or otherwise weakened (e.g., made more flexible) to define the hinges 165 (e.g., a living hinge). In some embodiments, the exit portion 170 can form a discontinuity such as a groove or the like that can form the hinges 165. Thus, each dilation member 164 can be configured to pivot and/or rotate about an axis (not shown) defined by the corresponding hinge 165.

Each dilation member 164 can be any suitable shape, size, and/or configuration. Similarly, the set of dilation members 164 can be any suitable arrangement. For example, while the set of dilation members 164 is shown in FIGS. 2 and 3 as including two dilation members 164, in other embodiments, the set of dilation members 164 can including a single dilation member, three dilation members, four dilation members, five dilation members, or more. The set of dilation members 164 forms a dilation surface 167. Although shown in FIG. 2 as being substantially flat, in other embodiments, the dilation surface 167 can be, for example, rounded, curved, tapered, dome-shaped or the like. More specifically, the dilation surface 167 can be arranged in such a manner as to reduce and/or substantially eliminate sharp corners and/or angles that can, in some instances, result in the sheath 160 scraping and/or becoming caught on a surface of the bodily tissue. Moreover, the dilation surface 167 can facilitate dilation, piercing and/or penetration of a bodily cavity. With the dilation members 164 forming, at the least, rounded corners, the exit portion 170 and/or the dilation members 164 can be advanced along a surface of the bodily tissue substantially without becoming caught thereon.

As shown in FIG. 2, the set of dilation members 164 can collectively form the dilation surface 167 when the sheath 160 and/or the set of dilation members 164 is in a first configuration. The dilation surface 167 can be, for example, a substantially closed surface. In this manner, the lumen 176 defined by the sheath 160 can be substantially isolated from a volume disposed in a distal position relative to the dilation members 164. In other embodiments, the dilation members 164 can collectively define an opening or the like when in the first configuration (not shown in FIG. 2). The sheath 160 can be transitioned from its first configuration to its second configuration by moving the insertion member 180 in the distal direction, as indicated by the arrow AA in FIG. 3. More particularly, with the implant 185 in contact with the insertion member 180, the distal movement insertion member 180 moves the implant 185 within the lumen 176 in the AA direction. As such, the implant 185 can contact the set of dilation members 164 to transition the each dilation member 164 from the first configuration to the second configuration, as indicated by the arrows BB in FIG. 3. Moreover, the dilation members 164 collectively define an opening 164 when in the second configuration through which the implant 185 can be advanced to expel the implant 185 from the lumen 176. Thus, the insertion member 180 can move the implant 185 in the distal direction (e.g., the AA direction) to place the implant 185 (e.g., an IUD) at a target location within the body (e.g., the fundus 14 of the uterus 11).

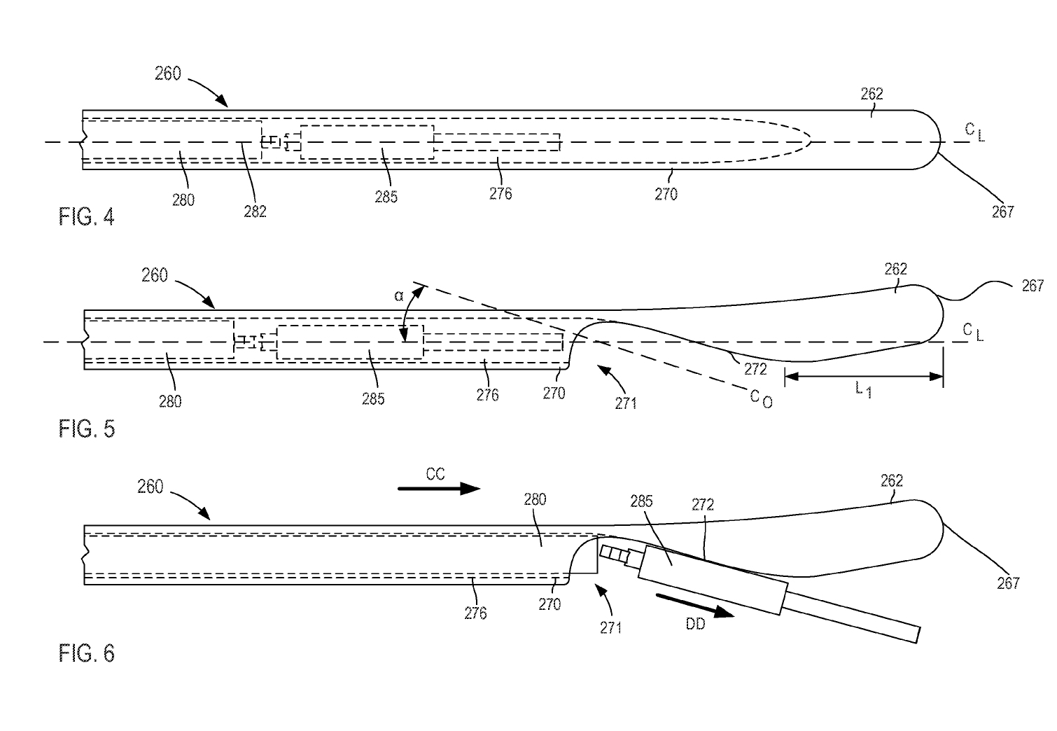

Although the sheath 160 is shown and described above as including the dilation members 164 and having the exit portion 170 disposed at the distal end of the sheath 160, in other embodiments, a sheath can be arranged its any suitable manner. For example, FIGS. 4-6 are schematic illustrations of a portion of a delivery device according to an embodiment. Such a delivery device can be used, for example, to deliver an implant 285 to a target location within the body. For example, in some embodiments, such a delivery device can be used to place an IUD in contact with the fundus 14 of or otherwise within the uterus 10 (see e.g., FIG. 1). The portion of the delivery device includes a sheath 260 that can be coupled to any suitable portion of a delivery device. In some embodiments, the sheath 260 can be movably coupled to and/or movably disposed within a portion of a handle or the like of a delivery device (not shown in FIGS. 4-6). Such a delivery device can include, for example, an actuator or the like that can be operable in moving the sheath 260 relative to the handle, as described above. In some embodiments, such an insertion device can include, for example a vacuum nozzle or the like that can be used to engage a portion of the cervix 12 and once engaged, a user can pull traction and/or can otherwise manipulate the cervix 12 to facilitate the insertion of the sheath 260 through the cervical os 13.

The sheath 260 can be formed from any suitable material or combination of materials such as, for example, those described above. More specifically, the sheath 260 can be formed or constructed from a substantially flexible material (e.g., a relatively high durometer rubber, siliconized rubber, polypropylene, polyethylene, and/or the like) that can allow for bending, twisting, opening, and/or otherwise reconfiguring of at least a portion of the sheath 260. For example, the sheath 260 can be sufficiently flexible to be advanced along a tortuous path defined by a portion of the body, yet can be sufficiently stiff to resist kinking, buckling, collapsing, and/or plastically deforming.

The sheath 260 includes at least distal end portion 262 and an exit portion 270, and defines a lumen 276. The lumen 276 movably receives a portion of an insertion member 280 having a distal end portion 282 that is configured to be placed in contact with the implant 285. For example, the implant 285 can be loaded into the sheath 260 to be movably disposed in the lumen 276 and similarly, the insertion member 280 can be inserted into the lumen 276 to be placed in contact with the implant 285. The distal end portion 262 of the sheath 260 is substantially solid and includes a distal surface that is spaced apart (by a distance L₁) from the lumen 276. Similarly stated, the lumen 276 does not extend through the distal (or dilation) surface 267 of the sheath 260, as described in further detail herein.

The distal end portion 262 of the sheath 260 can be any suitable shape, size, or configuration. For example, in some embodiments, the sheath 260 can be formed from a single material that can be extruded to form the sheath 260. In other embodiments, at least the distal portion 262 cats be co-extruded with the remaining portions of the sheath 260 wherein a second material can be introduced during an extrusion process to form the distal end portion 262 of the sheath 260 from a different material or blend of materials (e.g., the remaining portions of the sheath 260 are formed from a base material or the like and the distal end portion 262 is formed from the different material or the blend of materials). In still other embodiments, the distal end portion 262 can be, for example, over-molded about a portion of the sheath 260. Moreover, the sheath 260 can have a substantially constant outer diameter and/or inner diameter or can have an outer diameter and/or an inner diameter that is varied along a length of the sheath 260. For example, in some embodiments, the sheath 260 can include one or more discontinuities such as the hinges 165 (e.g., living hinges) described above with reference to the sheath 160.

The distal end portion 262 of the sheath can be formed from a substantially flexible material or blend of materials with a relatively low hardness (e.g., different from the material forming the sheath 260). For example, the distal end portion 262 can be formed from a relatively low durometer rubber, silicone, siliconized rubber, and/or the like that has a hardness (i.e., durometer) that is less than a hardness (i.e., durometer) of the sheath 260. In this manner, the relatively low hardness of the distal tip can, for example, limit and/or substantially prevent damage to bodily tissue as the sheath 260 is inserted into the body.

The exit portion 270 defines an opening 271 in communication with the lumen 276 and spaced apart by the distance L₁ from a distal surface of the sheath 260. More particularly, the exit portion 270 includes an exit surface 272 that defines, for example, an end portion of the lumen 276 and/or a portion of the opening 271. Thus, a distal portion of the exit surface 272 is spaced apart the distance L₁ from the distal surface of the sheath 260. As shown in FIG. 5, at least a portion of the lumen 276 defines a longitudinal centerline C_{L} extending therethrough. The opening 271 defined by the exit portion 270 extends through a circumferential (or side) surface of the sheath 260 and is, for example, laterally offset from the longitudinal centerline C_{L}. Furthermore, the opening 271 defines a centerline C_{O} that is transverse relative to the sheath 260 and defines an angle α, as shown in FIG. 5. In some embodiments, the angle α can be an acute angle. In this manner, the exit surface 272 can be separated from the distal surface 267 by the distance L₁ at or adjacent to an outer diameter of the sheath 260, and can be separated from the distal surface 267 by a second distance (not shown in FIG. 5) at or adjacent to an inner diameter of the sheath 260.

In use, the implant 285 can be loaded into sheath 260 via the opening 271. More specifically, the implant 285 can be moved along the opening centerline C_{O} in the proximal direction and can be in contact with the exit surface 272 in such a manner that the exit surface 272 guides the implant 285 in the proximal position to be disposed in the lumen 276 (FIG. 5). In other embodiments, the implant 285 can be loaded into the sheath 260 from the proximal end. Specifically, the insertion member 280 can be predisposed in the lumen 276 and/or can be inserted through a proximal portion (not shown) of the sheath 260 to place the distal end portion 282 in contact with the implant 285. With the distal end portion 282 of the insertion member 280 in contact wife the implant 285, the sheath 260 can be advanced through a bodily lumen or the like and disposed in a desired position relative to a target tissue (e.g., the fundus 14 of the uterus 11). For example, in some instances, the sheath 260 can be inserted through the cervical os 13 and further manipulated to place the opening 271 adjacent to a desired portion of the fundus. Moreover, the arrangement of the distal end portion 262 of the sheath 260 (e.g., being substantially solid and/or being formed from a material or blend of materials with a relatively small durometer) can facilitate the insertion of the sheath 260 through, for example, the cervical os 13 without being caught and/or causing damage to the surrounding tissue.

With the sheath 260 in the desired position (e.g., with the surface 267 against the fundus), the insertion member 280 can be advanced within the lumen 276, as indicated by the arrow CC in FIG. 6. In turn, the insertion member 280 can move the implant 285 in the CC direction (e.g., a distal direction along the longitudinal centerline C_{L} to place a portion of the implant 285 in contact with the exit surface 272. The arrangement of the exit surface 272 and the opening 271 is such that as the implant 285 is moved along the longitudinal centerline C_{L}, the exit surface redirects and/or otherwise guides the implant 285 toward the opening 271. That is to say, once the implant 285 is placed in contact with the exit surface 272, further distal movement of the insertion member 280 (or proximal movement of the sheath 160 relative to the insertion member 280) advances the implant 285 substantially in the direction of the opening centerline C_{O}, as indicated by the arrow DD in FIG. 6. Similarly, as the insertion member 280 is moved in the CC direction, a portion of the insertion member 280 can be placed in contact with the exit surface and in turn, can bend, flex, and/or otherwise deform in such a manner that at least the distal end portion 282 is substantially parallel with the opening centerline C_{O}, as shown in FIG. 6. Thus, the implant 285 can be advanced through the opening 271 to be conveyed from the lumen 276 to the target tissue (e.g., the fundus 14 of the uterus 11).

As shown in FIG. 6, the arrangement of the insertion member 280 can be such that as the implant 285 is advanced through the opening 271, the insertion member is maintained substantially within the sheath 260. In this manner, the likelihood of damage to the target tissue and/or a surrounding tissue caused by an excess in insertion force and/or an excess in distal movement of the insertion member 280 can be reduced or substantially eliminated. For example, in some embodiments, the exit portion 270 can define the opening 271 with a size and/or shape (e.g., a diameter) that is associated with and/or that corresponds to a size and/or shape of the implant 285, while being smaller than an size (e.g., diameter) of the insertion member 280. Thus, the insertion member 280 is prevented from being advanced in a distal direction to a position outside of the lumen 276.

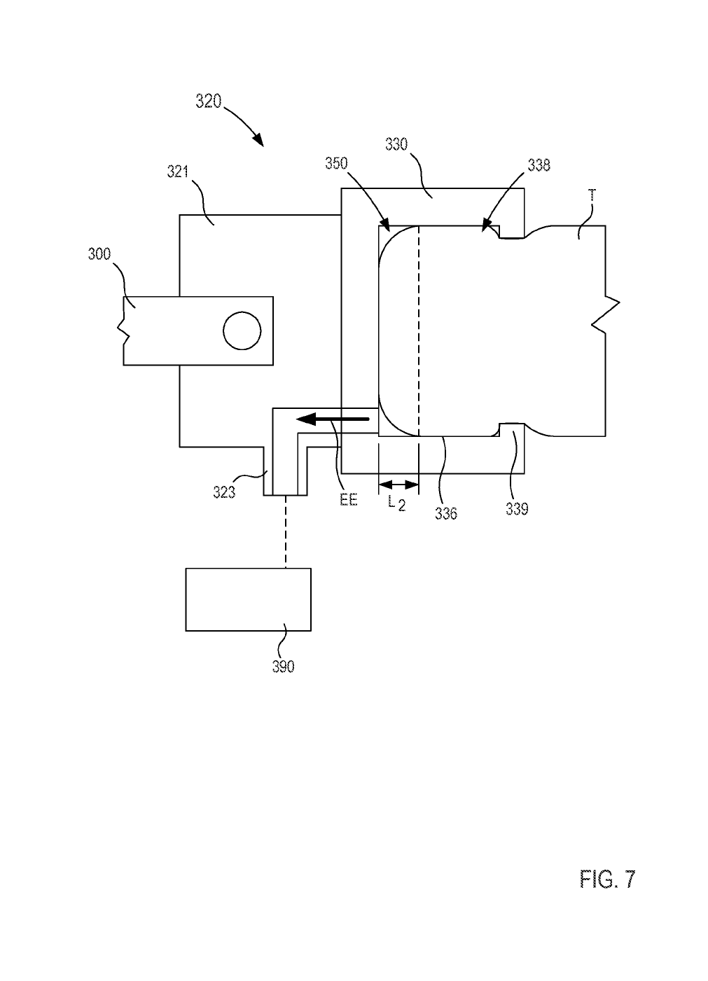

As described above, the sheaths 160 and 260 (or any of the sheaths described herein) can be included in and/or used with any suitable insertion device. In some embodiments, such an insertion device and/or tissue manipulation device can include a mechanism that can be placed in contact with a target tissue such as, for example, a portion of the cervix 12 (FIG. 1) and subsequently actuated to exert a suction force on the target tissue, thereby temporarily coupling the insertion device to the target tissue. With the device coupled to the target tissue, the insertion device can be manipulated to provide traction to the target tissue, which can facilitate the insertion and/or placement of the sheath 160 and/or 260 relative to the target tissue. By way of example, FIG. 7 is a schematic illustration of a vacuum nozzle 320 (also referred to as a vacuum head) coupled to a portion of a medical device 300 according to an embodiment. Although not shown in FIG. 7, the medical device 300 (also referred to herein as “device”) can be any suitable device configured to facilitate access to a bodily cavity during any suitable medical procedure and/or to facilitate the insertion of an implant, pharmaceutical, and/or the like. For example, in some embodiments, the device 300 can be substantially similar to those described in the '734 application and/or the '335 application incorporated by reference above. In some embodiments, the device 300 can be a tissue engagement device configured to engage and/or manipulate a target tissue within a bodily cavity.

As shown in FIG. 7, the vacuum nozzle 320 includes a connection portion 321 and an engagement portion 330. The connection portion 321 can be coupled to any suitable portion of the device 300 (e.g., a distal end portion of a retractor and/or handle of the device 300) for pivotal movement. For example, in some embodiments, the vacuum nozzle 320 (also referred to herein as “nozzle” or “head”) can be configured to move relative to the portion of the device 300 when disposed within a body cavity and/or attached to a target tissue (e.g., via a suction coupling). Thus, the movement can facilitate the insertion of a distal end portion of the device 300 into a portion of the body. In some embodiments, the coupling of the connection portion 321 of the nozzle 320 to the portion of the device 300 can be configured to define a range of motion (e.g., a pivoting and/or rotational motion) of the vacuum nozzle 320 relative to the portion of the device 300.

The connection portion 321 includes a vacuum port 323 (also referred to herein as “port”) that is in fluid communication with a vacuum source 390. The vacuum source 390 can be any suitable device, mechanism, assembly, etc. configured to produce a negative pressure differential once actuated. Although not shown in FIG. 7, the vacuum source 390 can be disposed within a portion of the device 300. For example, in some embodiments, the vacuum source 390 can be a syringe mechanism or the like disposed within a handle of the device 300, as described in the '335 application. In such embodiments, actuation of the vacuum source 390 can increase a volume within a syringe or the like, which in turn, reduces a pressure therein. Thus, the actuation of the vacuum source 390 produces a negative pressure differential between the port 323 and the vacuum source 390 that can result in a suction force being exerted within the port 323, as described in further detail herein.

As shown in FIG. 7, the engagement portion 330 of the nozzle 320 is coupled to the connection portion 321 and is configured to receive a portion of a target tissue T (as described in further detail herein). The engagement portion 330 can be any suitable shape, size, and/or configuration. For example, in some embodiments, the engagement portion 330 can be substantially cylindrical. More particularly, the engagement portion 330 can include and/or can otherwise be formed from, a set of annular walls. Thus, the engagement portion 330 includes an inner surface 336 that defines a suction volume 338 that is in fluid communication with the port 323. For example, the engagement portion 330 can define an opening that can place the suction volume 338 in fluid communication with the port 323 when the engagement portion 330 is coupled to the connection portion 321. In this manner, when the vacuum source 390 is actuated, a negative pressure (e.g., a suction force) is produced within the suction volume that can be operable in retaining the target tissue T within at least a portion of the suction volume 338, as described in further detail herein.

The inner surface 336 also includes a rib 339 disposed at a distal end of the engagement portion 330. The rib 339 can be, for example, a protrusion, a tab, a ridge, a rail, a flange, a ring, and/or like that extends from the inner surface 336 into the suction volume 338. In some embodiments, the rib 339 can be substantially continuous (e.g., continuously encompasses the suction volume 338). In other embodiments, the rib 339 can include multiple portions and/or sections, defining one or more channels therebetween. As such, the rib 339 can extend from the inner surface 336 to selectively engage a portion of the target tissue T when the target tissue T is disposed in the suction volume 338, as described in further detail herein. Although the rib 339 is shown in FIG. 7 as being substantially rectangular, in other embodiments, the rib 339 can be any suitable shape or size. For example, in some embodiments, the rib 339 can include a first portion (e.g., a distal portion) that is substantially rounded and a second portion (e.g., a proximal portion) that is substantially linear. Moreover, while the rib 339 is shown in FIG. 7 as extending in a substantially perpendicular direction from the inner surface 336, in some embodiments, at a proximal surface of the rib 339 can be in-cut or the like. That is to say, a width of the rib 339 can increase as the rib 339 extends from the inner surface 336.

In use, at least a portion of the device 300 can be inserted into a body cavity and manipulated to place the head 320 in contact with the target tissue T. For example, in some instances, at least a portion of the device 300 can be inserted into the vagina 15 of a patient and advanced to place the head 320 in contact with a portion of the cervix 12 (i.e., the target tissue T). Once in contact with the target tissue T, the vacuum source 390 can be actuated and in turn, a suction force can be produced in the port 323 of the connection portion 321 and the suction volume 338 of the engagement portion 330, as indicated by the arrow EE in FIG. 7. Thus, the suction force produced within the suction volume 338 can draw a portion of the target tissue T into the suction volume 338.

In some embodiments, the portion of the target tissue T can be selectively placed in contact with the inner surface 336 of the engagement portion 330 when drawn into the suction volume 338. For example, as shown in FIG. 7, the target tissue T can be drawn into the suction volume 338 and selectively into contact with the inner surface 336 to define a vacuum pathway 350 between a first portion of the target tissue T and a portion of the inner surface 336. More specifically, in some embodiments, the inner surface 336 can include a proximal portion (e.g., associated with a proximal wall or the like) and a circumferential portion (e.g., associated with the set of annular walls, described above) forming an intersection portion therebetween. In some embodiments, the intersection portion formed between the proximal portion and the circumferential portion can define a curved shape having a predetermined radius of curvature. The radius of curvature defined by the intersection portion can, for example, be sufficiently small such when fee target tissue T is drawn into the suction volume 338 and selectively placed in contact with the inner surface 336, the intersection portion is spaced apart a distance L₂ from a surface of the target tissue T, thereby defining the vacuum pathway 350. In some embodiments, the vacuum pathway 350 can be a substantially continuous volume that circumscribes a portion of the target tissue T and that can maintain substantially continuous communication with the port 323. As such, the suction force produced by the vacuum source 390 can be substantially consistent within the vacuum pathway 350 and can, for example, be distributed with substantial uniformity about the portion to the target tissue T.

As shown in FIG. 7, the target tissue T can be drawn into the suction volume 338 such that a surface of the rib 339 is placed in contact with a surface of the target tissue T. More specifically, since the rib 339 extends from the inner surface 336 (as described above), the rib 339 can define a diameter that is smaller than a diameter of the remaining portions of inner surface 336. Thus, with the target tissue T disposed in the suction volume 338 and selectively in contact with the inner surface 336, the rib 339 can deform a corresponding portion of the target tissue T and as such, can place at least a portion of the proximal surface of the rib in contact with the target tissue T. Accordingly, the contact between the proximal surface of the rib 339 and the target tissue T can limit movement of the target tissue T in a direction away from the connection portion 321 (i.e., the distal direction). In this manner, the suction force exerted on a first portion of the target tissue T via the vacuum pathway 350 and the contact between at least the proximal surface of the rib 339 and a second portion of the target tissue T can retain the target tissue T within the engagement portion 330 (e.g., the suction volume 338). Moreover, the target tissue T can be retained within the engagement portion with a desired force sufficient to substantially prevent the target tissue T from being withdrawn from the engagement portion 330 when the device 300 is manipulated to exert a traction force on the target tissue T. As such, the target tissue T can be manipulated, moved, and/or otherwise reoriented to facilitate the insertion of, for example, an implant or the like. For example, in some instances, the nozzle 320 can retain a portion of the cervix 12 in the engagement portion 330 (e.g., the suction volume 338), while a traction force is applied thereto, thereby facilitating access (e.g., for a sheath or other delivery mechanism) through the cervical os 13 and into the uterus 11.

In some embodiments, the connection portion 321 can include one or more surfaces and/or portions that can be formed from a substantially transparent or translucent that can, for example, allow for visualization of at least a portion of the suction volume 338. In this manner, a user can determine, for example, if a suitable portion of the target tissue T is disposed within the suction volume 338 and/or in contact with the inner surface 336. In some embodiments, such a surface and/or portion can be shaped as a lens or the like that can magnify an appearance of, for example, a portion of the target tissue T disposed in the suction volume.

In some embodiments, an amount of suction force exerted on the target tissue T can be increased or decreased by changing the arrangement of the engagement portion 330. For example, in some embodiments, the size of the rib 339 can be increased or decreased to increase or decrease, respectively, a contact surface between the portion of the target tissue T and, for example, the proximal surface of the rib 339. Accordingly, an increase in a size of the contact surface can, for example, result in an increase in a force configured to resist the distal movement of the target tissue T relative to the engagement portion 330 (as described above) without a need, for example, to increase a suction force (e.g., an increase in a negative pressure differential produced by the vacuum source 390).

In a similar manner, an increase in a volume of the suction volume 338 and/or the vacuum pathway 350 can increase a force exerted on the target tissue T to retain the vacuum nozzle (or head) 320 in contact with the target tissue T at higher pull forces. Thus, by increasing the volume of the suction volume 338 and/or the vacuum pathway 350 a suction force as result of a negative pressure differential produced by the vacuum source 390 can be reduced, while still retaining the target tissue T within the engagement portion 330 during traction. For example, in some embodiments, a cross-sectional area of the inner surface 336 can be increased or decreased to increase or decrease, respectively, a force to retain the target tissue T in the engagement portion 330. in other embodiments, a depth of the suction volume 338 can be increased or decreased to increase or decrease, respectively, a force to retain the target tissue T in the engagement portion 330. In this other embodiments, the radius of curvature defined by the transition portion of the inner surface can be increased or decreased to increase or decrease, respectively, a force to retain the target tissue T in the engagement portion 330.

Although the vacuum nozzle 320 is shown in FIG. 7 as including a single vacuum port 323, in other embodiments, a nozzle can include any number of vacuum ports. The vacuum ports can be independent of one another or connected in parallel or in series, and can be arrangement, in any suitable orientation relative to an engagement portion. Moreover, the inner surface 336 can be configured to define one or more channels or the like that can be in fluid communication with the vacuum pathway 350. In some embodiments, the head 320 can include an elongate member or the like that can extend in a distal direction beyond a distal surface of the engagement portion 330. In such embodiments, the elongate member can, for example, be disposed in the cervical os 13 or the like and can receive an implant that can be moved therethrough to be placed at a target location such as, for example, the fundus 14 of the uterus 11.

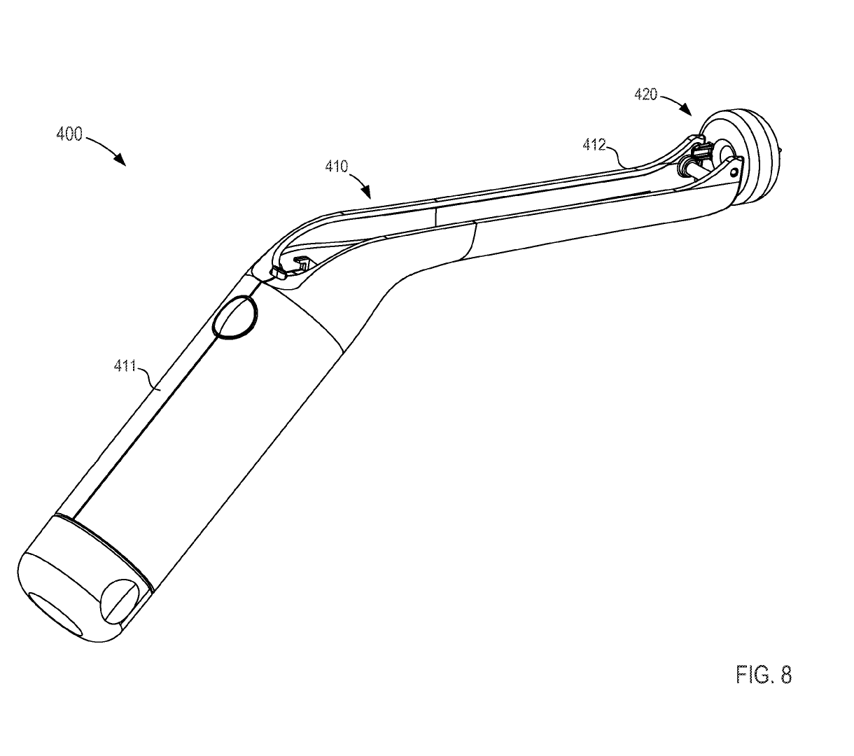

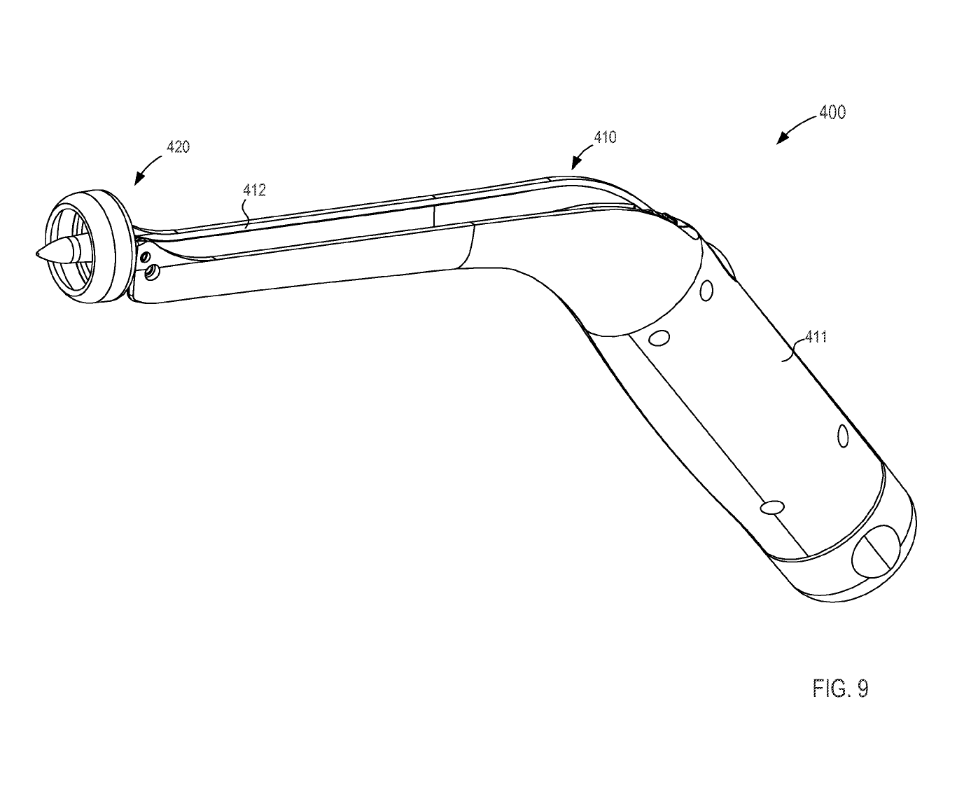

FIGS. 8-13 illustrate a portion of a medical device 400 according to an embodiment. The medical device 400 (and any of the other insertion devices and/or tissue engagement devices described herein) can be used with any of the sheaths, insertion members or the like described herein. In some embodiments, the medical device 400 can be used to engage, manipulate and/or secure a bodily tissue to facilitate a procedure on the bodily tissue. The medical device 400 (also referred to herein as “device”) includes a retractor 410 and a vacuum nozzle 420. The vacuum nozzle 420 can be coupled to the retractor 410 for pivotal movement, as described in further detail herein. As shown in FIGS. 8 and 9, the refractor 410 includes a proximal end portion 411 and a distal end portion 412, The proximal end portion 411 can be engaged by a user to manipulate the retractor 410, The distal end portion 412 can be movably coupled to fee vacuum nozzle 420, as described in further detail herein. The retractor 410 can be, for example, substantially similar to or the same as any of the retractors, body portions, housings, and/or delivery devices described in the '734 application and/or '335 application incorporated by reference above. As such, fee retractor 410 is not described in further detail herein.

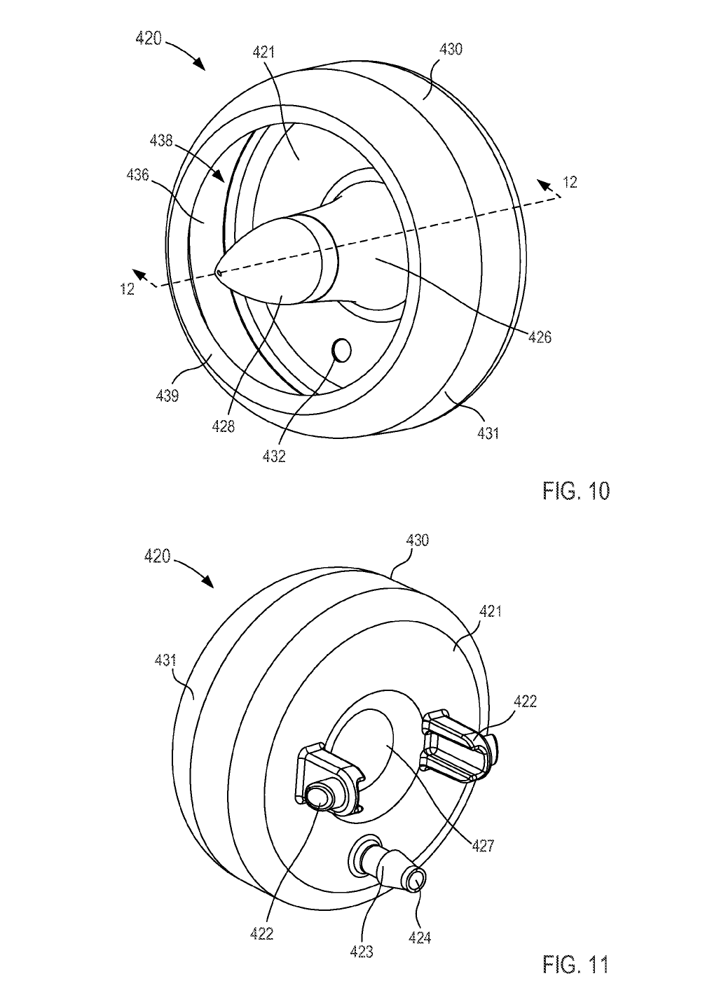

As shown in FIGS. 10-13, the vacuum nozzle 420 (also referred to herein as “nozzle” or “head”) includes a connection portion 421 and an engagement portion 430. The connection portion 421 includes a vacuum port 423 (also referred to herein as “port”) and a set of connection members 422. The connection members 422 are configured to be movably coupled to the distal end portion 412 of the retractor 410. For example, as shown in FIG. 11, the connection members 422 can include substantially cylindrical protrusions or the like that can be disposed in a corresponding opening defined by the distal end portion 412 of the retractor to movably couple the nozzle 420 to the distal end portion 412 of the retractor 410 (see e.g., FIGS. 8 and 9). In this manner, the nozzle 420 can be configured to move relative to the retractor 410, For example, in some embodiments, the nozzle 420 can be configured to move relative to the retractor 410 when disposed within a body cavity and/or attached to a target tissue (e.g., via suction coupling). Thus, the movement can facilitate the insertion of the distal end portion 412 of the retractor 410. In some embodiments, the nozzle 420 can be configured to rotate relative to the retractor 410. In such embodiments, the coupling of the nozzle 420 to the retractor 410 can define a range of motion of the nozzle 420 relative to the retractor 410 (e.g., the retractor 410 and/or the nozzle 420 can include any number of stops, channels, guides, tabs, flanges, pivot points, etc. configured to control, direct, or otherwise influence movement of the nozzle relative to the handle 410).

The port 423 of the connection portion 421 defines a lumen 424 in fluid communication with a vacuum source (not shown in FIGS. 8-13) and a portion of the engagement portion (see e.g., FIG. 8). The vacuum source can be any suitable device, mechanism, assembly, etc. configured to produce a negative pressure differential once actuated. For example, in some embodiments, the vacuum source can be a syringe mechanism or the like disposed within a handle of the retractor 410, as described in the '335 application. In such embodiments, actuation of the vacuum source can increase a volume within a syringe or the like, which in turn, reduces a pressure therein. Thus, the actuation of the vacuum source produces a negative pressure differential between the port 423 and the vacuum source that can result in a suction force being exerted within the lumen 424, as described in further detail herein.

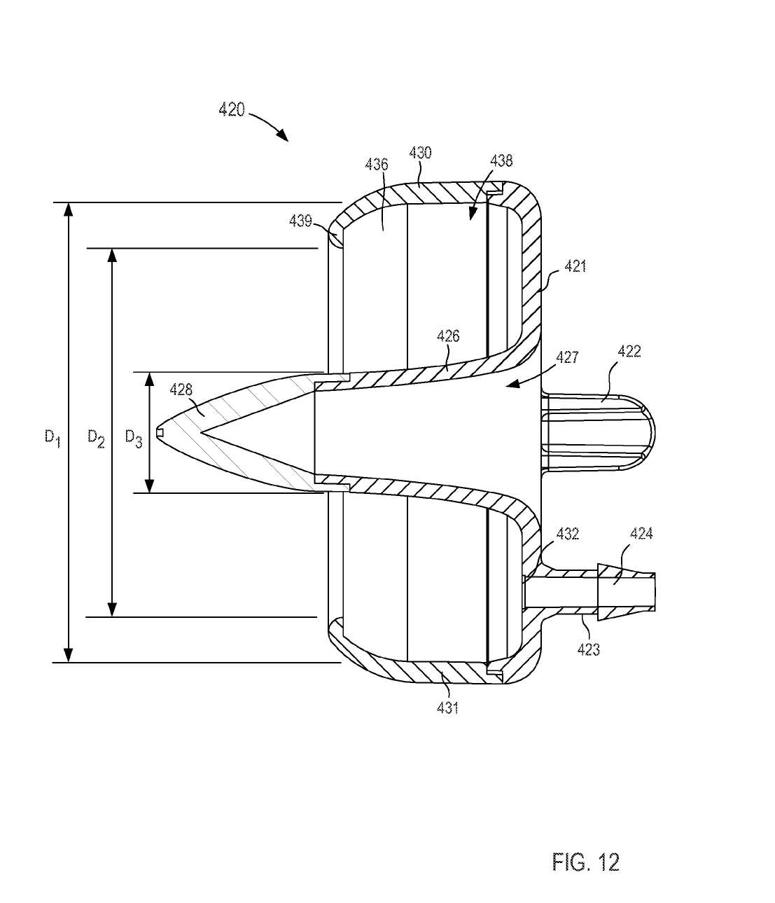

As shown in FIGS. 10-13, the engagement portion 430 of the nozzle 420 is coupled to the connection portion 421 and is configured to receive a portion of a target tissue (as described in further detail herein). The engagement portion 430 can be any suitable shape, size, and/or configuration. For example, in some embodiments, the engagement portion 430 can be substantially cylindrical including and/or otherwise being formed from a set of annular walls 431. As such, the annular walls 431 include an inner surface 436 having a diameter D₁. The inner surface 436 defines a suction volume 438 configured to be in fluid communication with the suction port 423. More specifically, the engagement portion 430 defines an opening 432 that places the suction volume 438 in fluid communication with the lumen 424 of the port 423, as shown in FIG. 12. In this manner, when the vacuum source is actuated, a negative pressure (e.g., a suction force) is produced within the suction volume 438 that can be operable in retaining a target tissue within at least a portion of the suction volume 438, as described in further detail herein.

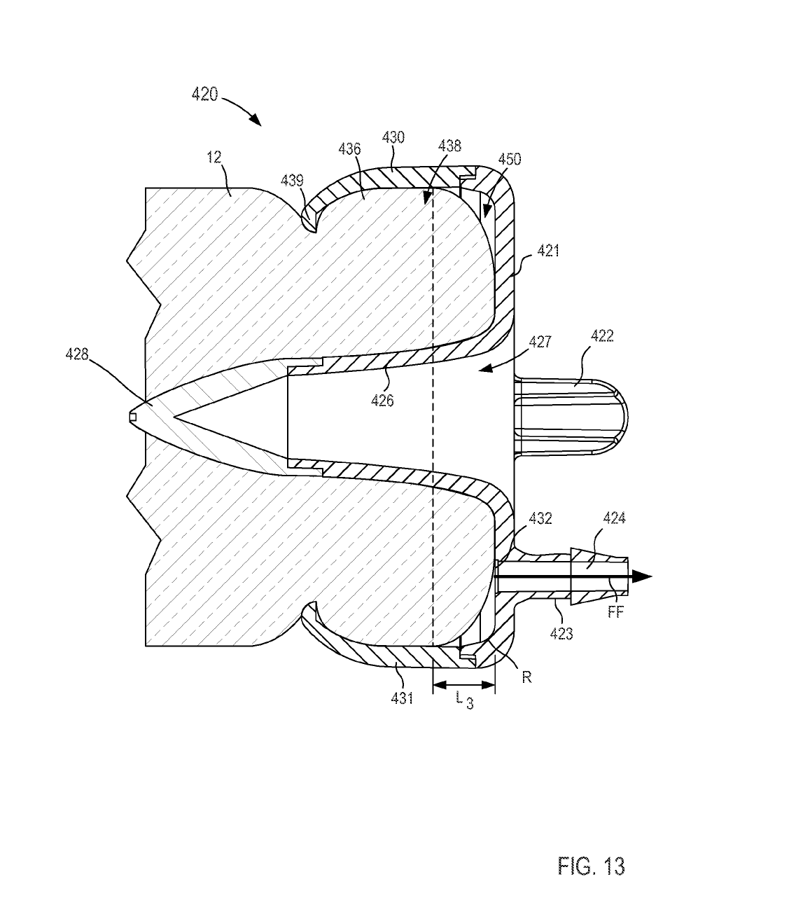

The inner surface 436 also includes and/or forms a rib 439 disposed at a distal end of the set of annular walls 431. The rib 439 can be, for example, a protrusion, a tab, a ridge, a rail, a flange, a ring, and/or like that extends from the inner surface 436 into the suction volume 438. For example, as shown in FIG. 12, the rib 439 has a diameter D₂ that is smaller than the diameter D₁ defined by the inner surface 436 (e.g., associated with the suction volume 438). As such, the rib 439 can extend from the inner surface 436 to selectively engage a portion of the target tissue when disposed in the suction volume 438 (see e.g., FIG. 13). More specifically, the diameter D₂ of the rib 439 can be such that the rib 439 deforms a portion of the target tissue when disposed in the suction volume 438, which can be operable in retaining a target tissue within the suction volume 438.

In some embodiments, the rib 439 can be substantially continuous (e.g., continuously encompasses the suction volume 438). In other embodiments, the rib 439 can include multiple portions and/or sections, defining one or more channels therebetween. The rib 439 can be any suitable shape or size. For example, in this embodiment, a distal surface of the rib 439 is substantially rounded, while a proximal surface of the rib 439 is substantially linear. Moreover, while the rib 439 is shown in FIG. 12 as extending in a substantially perpendicular direction from the inner surface 436, in some embodiments, the rib 439 can extend from the inner surface 436 at any suitable angle. Moreover, in some embodiments, the proximal surface of the rib 439 can be in-cut or the like, wherein a width of the rib 439 increases as the rib 439 extends irons the inner surface 436.

The engagement portion 430 also includes an elongate portion 426 that extends from a proximal end portion of the engagement portion 430. As shown, the elongate portion 426 extends substantially though a center of the engagement portion 430. In other embodiments, an elongate portion can be offset from a center of an engagement portion. The elongate portion 426 can be any suitable shape, size, or configuration. For example, as shown in FIGS. 10, 12, and 13, the elongate portion 426 can be substantially tapered (e.g., tapered to a rounded distal end). Said a different way, the elongate portion 426 can have a diameter D₃ that decreases as the elongate portion 426 extends in the distal direction. In other embodiments, the elongate portion 426 need not be tapered. At least a portion of the elongate portion 426 can be monolithically formed with the vacuum nozzle 420. For example, the vacuum nozzle 420 can be a single molded piece. As such, the elongate portion 426 can have a stiffness that is sufficiently large to allow for insertion into a body cavity without undue deformation. For example, the elongate portion 426 can be sufficiently stiff as to resist and/or withstand an axial force exerted thereon when the elongate portion 426 enters the cervix os or other body cavity.

The elongate portion 426 defines a lumen 427 that is configured to receive, for example, a sheath (e.g., the sheath 160 and/or 260), an implant, a pharmaceutical, and/or any suitable portion of an insertion mechanism such as a catheter, a tube, a rod, an instrument, and/or the like. In this manner, the elongate portion 426 can allow an implant, pharmaceutical, etc. to be advanced through the suction volume 438 to be delivered to a desired portion of the body that can be, for example, in a distal position relative to the nozzle 420. Moreover, the elongate member 426 includes a distal tip 428 for dilation member) that is at least partially disposed is a distal position relative to the rib 439 (see e.g., FIG. 12). In some embodiments, the distal tip 428 can be formed independently from the elongate portion 426 and coupled thereto. For example, the distal tip 428 can be formed from a material (e.g., silicone, siliconized rubber, rubber, and/or the like) having a durometer that is less than a durometer associated with the material forming the elongate member 426, the rib 439 and/or any other portion of the nozzle 420. Thus, the reduced durometer can, for example, allow the distal tip 428 to bend, flex, and/or otherwise deform in response to the axial force (described above), and thus, can reduce and/or substantially eliminate damage to bodily tissue during insertion of the nozzle 420. Although not shown in FIGS. 12 and 13, the distal tip 428 can be transitioned from a first configuration (e.g., a closed configuration as shown in FIG. 12) and a second configuration (e.g., an open configuration in which the lumen 427 extends therethrough). For example, in some embodiments, the distal tip 428 can include one or more dilation members or the like that can be transitioned from the first configuration, in which the dilation members are substantially closed, to the second configuration, in which the dilation members are substantially open. In some embodiments, the distal tip 428 can define one or more slits, cuts, openings, notches, and/or the like that can, for example, form at least a portion of the dilation members. Thus, a sheath, implant, pharmaceutical, and/or any other suitable portion of the insertion mechanism or the like can be passed through the lumen 427 to be delivered to a desired bodily tissue (e.g., the fundus 14 of the uterus 11 (FIG. 1)), as described above.

In use, at least a portion of the device 400 can be inserted into a body cavity and manipulated to place the nozzle 420 in contact with a target tissue. For example, in some instances, the distal end portion 412 of the retractor 410 can be inserted into the vagina 15 of a patient and advanced to place the nozzle 420 in contact with a portion of the cervix 12 (i.e., the target tissue). Once in contact with the cervix 12 (as shown in FIG. 13), the vacuum source can be actuated and in turn, a suction force can be produced in the lumen 424 of the port 423 and at least a portion of the suction volume 438 of the engagement portion 430, as indicated by the arrow FF in FIG. 13. Thus, the suction force produced within the suction volume 438 can draw a portion of the cervix 12 into the suction volume 438. Moreover, with the portion of the cervix 12 drawn into the suction volume 438, the elongate portion 426 can extend through the cervical os (not shown in FIG. 13) such that at least a portion of the distal tip 428 is positioned within the uterus (not shown in FIG. 13).

In some embodiments, the portion of the cervix 12 can be selectively placed in contact with the inner surface 436 of the engagement portion 430 when drawn into the suction volume 438. For example, as shown in FIG. 13, the cervix 12 can be drawn into the suction volume 438 and selectively placed into contact with the inner surface 436 to define a vacuum pathway 450 between a first portion of the cervix 12 and a portion of the inner surface 436. More specifically, in some embodiments, the inner surface 436 can include a proximal portion (e.g., associated with a proximal wall or the like) and a circumferential portion (e.g., associated with the set of annular walls, described above) forming an intersection portion therebetween. In some embodiments, the intersection portion formed between the proximal portion and the circumferential portion can define a curved shape having a predetermined radius of curvature R, as shown in FIG. 13. The radius of curvature R defined by the intersection portion can, for example, be sufficiently small such when the cervix 12 is drawn into the suction volume 438 and selectively placed in contact with the inner surface 436, the intersection portion is spaced apart a distance L₃ from a surface of the cervix 12, thereby defining the vacuum pathway 450. In some embodiments, the vacuum pathway 450 can be a substantially continuous volume that circumscribes a portion of the cervix 12 and that can maintain substantially continuous communication with the port 423. As such, the suction force produced by the vacuum source 490 can be substantially consistent within the vacuum pathway 450 and can, for example, be distributed with substantial uniformity about the portion to the cervix 12. Moreover, while the cervix 12 is shown in FIG. 13 as being in contact with the elongate portion 426 at or adjacent to the proximal portion of the inner surface 426, in other embodiments, the arrangement of the elongate portion 426 can be such that the cervix 12 is similarly spaced apart from the a portion of the elongate portion 426.

As shown in FIG. 13, the cervix 12 can be drawn into the suction volume 438 such that a surface of the rib 439 is placed in contact with a surface of the cervix 12. More specifically, since the rib 439 extends from the inner surface 436 (as described above), the rib 439 can define a diameter that is smaller than a diameter of the remaining portions of inner surface 436. Thus, with the cervix 12 disposed in the suction volume 438 and selectively in contact with the inner surface 436, the rib 439 can deform a corresponding portion of the cervix 12 and as such, can place at least a portion of the proximal surface of the rib in contact with the cervix 12. Accordingly, the contact between the proximal surface of the rib 439 and the cervix 12 can limit movement of the cervix 12 in a direction away from the connection portion 421 (i.e., the distal direction). In this manner, the suction force exerted on a first portion of the cervix 12 via the vacuum pathway 450 and the contact between at least the proximal surface of the rib 439 and a second portion of the cervix 12 can retain the cervix 12 within the engagement portion 430 (e.g., the suction volume 438). Moreover, the cervix 12 can be retained within the engagement portion with a desired force sufficient to substantially prevent the cervix 12 from being withdrawn from the engagement portion 430 when the device 400 is manipulated to exert a traction force on the cervix 12. As such, the cervix 12 can be manipulated, moved, and/or otherwise reoriented to facilitate the insertion of, for example, an implant or the like. For example, in some instances, the nozzle 420 can retain a portion of the cervix 12 in the engagement portion 430 (e.g., the suction volume 438), while a traction force is applied thereto, thereby facilitating access (e.g., for a sheath or other delivery mechanism) through the cervical os 13 and into the uterus 11.

In some embodiments, an amount of suction force exerted on the cervix 12 can be increased or decreased by changing the arrangement of the engagement portion 430. For example, in some embodiments, the size of the rib 439 can be increased or decreased to increase or decrease, respectively, a contact surface between the portion of the cervix 12 and, for example, the proximal surface of the rib 439. For example, in some embodiments, the diameter D₂ defined by the rib 439 can be decreased. Accordingly, an increase in a size of the contact surface can, for example, result in an increase in a force configured to resist the distal movement of the cervix 12 relative to the engagement portion 430 (as described above) without a need, for example, to increase a suction force (e.g., an increase in a negative pressure differential produced by the vacuum source).

In a similar manner, an increase in a volume of the suction volume 438 and/or the vacuum pathway 450 can increase a force exerted on the cervix 12 to retain the vacuum nozzle 420 in contact with the cervix 12 at higher pull forces. Thus, by increasing the volume of the suction volume 438 and/or the vacuum pathway 450 a suction force as result of a negative pressure differential produced by the vacuum source 490 can be reduced, while still retaining the cervix 12 within the engagement portion 430 during traction. For example, in some embodiments, a cross-sectional area of the suction volume 438 can be increased or decreased to increase or decrease, respectively, a force to retain the cervix 12 in the engagement portion 430. By way of example, in some embodiments, the diameter D₃ of the elongate portion 426 can be decreased and as such, the suction volume 438 defined between the elongate portion 426 and the inner surface 436 can be increased. In other embodiments, a depth of the suction volume 438 can be increased or decreased to increase or decrease, respectively, a force to retain the cervix 12 in the engagement portion 430. In this other embodiments, the radius of curvature defined by the transition portion of the inner surface can be increased or decreased to increase or decrease, respectively, a force to retain the cervix 12 in the engagement portion 430.

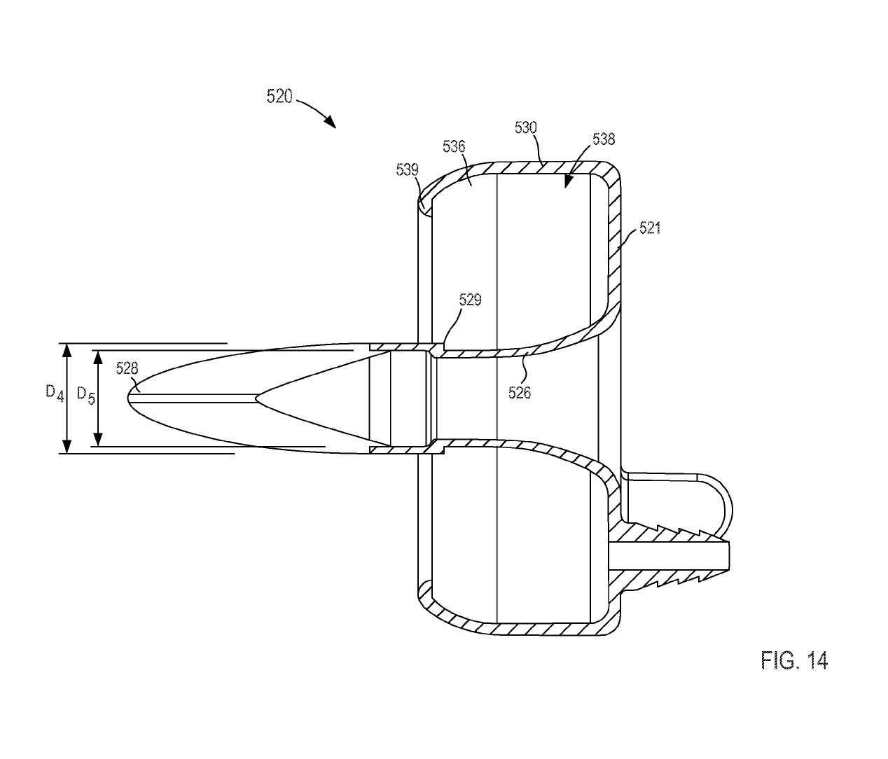

Although the vacuum nozzle 420 is shown in FIGS. 10-13 as including multiple pieces that axe joined together, in other embodiments, the vacuum nozzle (or head) 420 (and any of the heads described herein) can be monolithically constructed. Although the vacuum nozzle 420 is shown in FIGS. 10-13 as including the elongate portion 426 with the diameter D₅ that forms a substantially smooth taper as the elongate portion 426 extends in the distal direction, in other embodiments, a vacuum nozzle can include an elongate portion that can form, for example, a rib or the like. By way of example, FIG 14 is an illustration of a vacuum nozzle 520 according to another embodiment. The vacuum nozzle 520 includes a connection portion 521 and an engagement portion 530. The vacuum nozzle 520 can be substantially similar in form and/or function as the vacuum nozzle 420 described above with reference to FIGS. 8-13. Thus, aspects of the vacuum nozzle 520 that are similar to corresponding aspects of the vacuum nozzle 420 are not described in further detail herein. The vacuum nozzle 520 can differ from the vacuum nozzle 420, however, in the arrangement of the engagement portion 530. More specifically, as shown in FIG. 14, the engagement portion 530 includes an inner surface 536 that includes and/or forms a first rib 539 and that defines a suction volume 538 (e.g., similar to the rib 439 and the suction volume 438, respectively, included in the vacuum nozzle 420). The engagement portion 530 also includes an elongate portion 526 that extends from a proximal portion of the inner surface 536 and that is coupled to a distal tip 528 (e.g., similar to the distal tip 428 of the vacuum nozzle 420). As shown, the elongate portion 526 includes a second rib 529 that extends from a surface of the elongate portion 526 toward the first rib 539. In this manner, the second rib 529 can have a first diameter D₄ that is greater than a second diameter D₅ of at least a portion of the elongate portion 526 that is proximal to the second rib 529. Thus, the first rib 539 and the second rib 529 can be configured to collectively engage a portion of a target tissue when the target tissue is disposed in the suction volume 538, as described in detail above with reference to the nozzle 420.

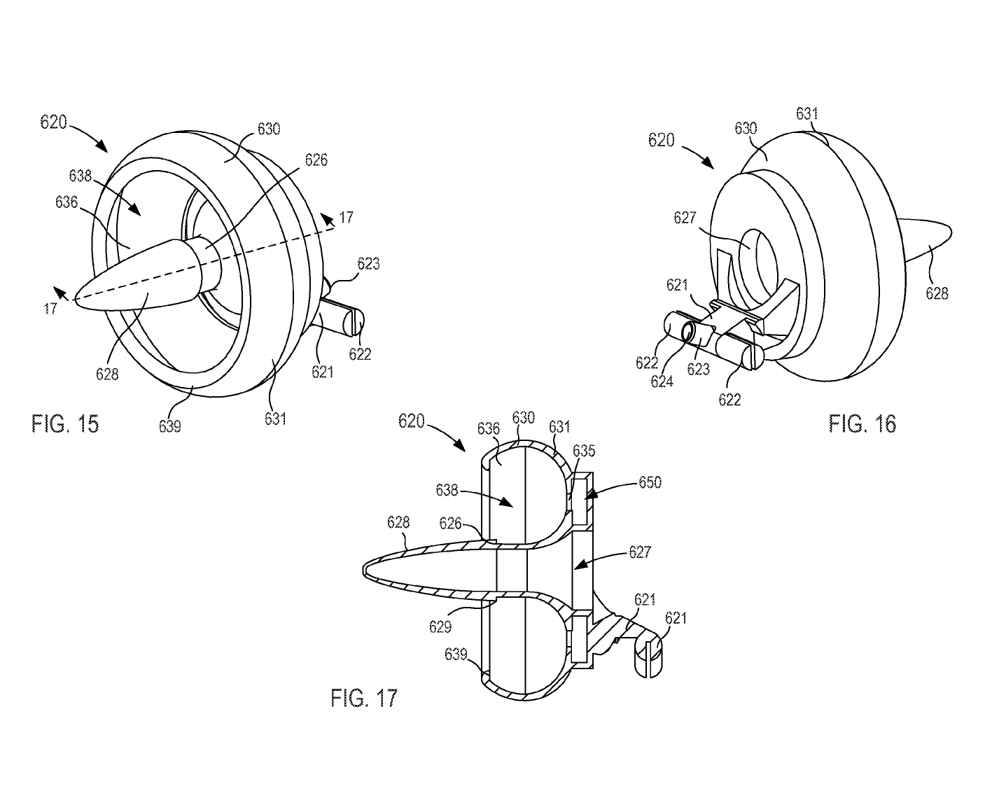

FIGS. 15-17 illustrate a vacuum nozzle 620 (also referred to herein as “nozzle” or “head”) according to another embodiment. The nozzle 620 can be used with any suitable device (i.e., an insertion device and/or a tissue engagement device) such as, for example, the retractor 410 described with reference to FIGS. 8 and 9. The nozzle 620 includes a connection portion 621 and an engagement portion 630. Aspects of the vacuum nozzle 620 can be substantially similar in form and/or function as the vacuum nozzle 420 described above with reference to FIGS. 8-13. Thus, such aspects of the vacuum nozzle 620 that are similar to corresponding aspects of the vacuum nozzle 420 are not described in further detail herein.

The connection portion 621 of the nozzle 620 includes a vacuum port 623 (also referred to herein as “port”) and a set of connection members 622. The connection members 622 are configured to be movably coupled to a portion of the device, as described above. In this manner, the nozzle 620 can be configured to move relative to the device when disposed within a body cavity and/or attached to a target tissue (e.g., via suction coupling). Thus, the movement can facilitate the insertion of a portion of the device. The port 623 of the connection portion 621 defines a lumen 624 in fluid communication with a vacuum source (not shown in FIGS. 15-17) and a portion of the engagement portion 630 (see e.g., FIG. 17). The vacuum source can be any suitable device, mechanism, assembly, etc. configured to produce a negative pressure differential once actuated, as described in detail above with reference to the nozzle 420 in FIGS. 8-13.

As shown in FIGS. 15 and 17, the engagement portion 630 of the nozzle 620 is coupled to the connection portion 621 and is configured to receive a portion of a target tissue (as described in further detail herein). The engagement portion 630 can be any suitable shape, size, and/or configuration. For example, in some embodiments, the engagement portion 630 can be substantially cylindrical including and/or otherwise being formed from a set of annular walls 631. As such, the annular walls 631 include an inner surface 636 that defines a suction volume 638 configured to be in fluid communication with the suction port 623. In this manner, when the vacuum source is actuated, a negative pressure (e.g., a suction force) is produced within the suction volume 638 that can be operable in retaining a target tissue within at least a portion of the suction volume 638, as described in further detail herein.

The inner surface 636 also includes and/or forms a rib 639 disposed at a distal end of the set of annular walls 631 and defines a vacuum pathway 650 disposed at or adjacent to a proximal end portion of the set of annular walls 631. The rib 639 can be, for example, a protrusion, a tab, a ridge, a rail, a flange, a ring, and/or like that extends from the inner surface 636 into the suction volume 638. In some embodiments, the rib 639 can be substantially similar to the rib 439 of the nozzle 420, as described in detail above with reference to FIG. 12. For example, the rib 639 can extend from the inner surface 636 to selectively engage a portion of the target tissue when disposed in the suction volume 638. More specifically, the rib 639 can be configured to engage and/or deform a portion of the target tissue when disposed in the suction volume 638, which can be operable in retaining a target tissue within the suction volume 638, as described above with reference to the nozzle 420.

The vacuum pathway 650 can be any suitable configuration. For example as shown in FIG. 17, the engagement portion 630 can define an annular channel or the like to form and/or define the vacuum pathway 650. While the vacuum pathway 650 and/or the part of the engagement portion 630 defining the vacuum pathway 650 is substantially rectangular (in cross-sectional shape), in other embodiments, the vacuum pathway 650 can have any suitable shape such as, for example, oblong, elliptical, circular, square, pentagonal, octagonal, etc. In addition, the engagement portion 630 defines an annular opening 635 that places the vacuum pathway 650 in fluid communication with the suction volume 638. Although not shown in FIGS. 15-17, the vacuum pathway 650 is in fluid communication with the lumen 624 defined by the port 623. As such, a negative pressure produced by the vacuum source can result in a suction force within the vacuum pathway 650, as described in further detail herein. More specifically, the vacuum pathway 650 and the annular opening 635 substantially circumscribe a proximal surface of the engagement portion 630 and thus, define a continuous pathway about a portion of the target tissue (when disposed in the suction volume 638) and that can maintain substantially continuous communication with the port 623. As such, the suction force produced by the vacuum source can be substantially consistent (e.g., spatially uniform) within the vacuum pathway 650 and the annular opening 635 and, in turn, the suction force can be distributed with substantial uniformity about the portion to the target tissue. Furthermore, by defining a channel that is in continuous communication with the port and the suction volume, the target tissue can be disposed within the suction volume 638 and in contact with the inner surface 636 without having a portion that is spaced apart, as described above with reference to the nozzle 420.

The engagement portion 630 also includes an elongate portion 626 that extends from a proximal end portion of the engagement portion 630. As shown, the elongate portion 626 extends substantially though a center of the engagement portion 630. In other embodiments, an elongate portion can be offset from a center of an engagement portion. The elongate portion 626 can be any suitable shape, size, or configuration. For example, as shown in FIGS. 15 and 17, the elongate portion 626 can be substantially tapered (e.g., tapered to a rounded distal end). More specifically, the elongate portion 626 can be substantially similar to or the same as the elongate portion 526 described above with reference to FIG. 14. In this manner, the elongate portion 626 includes a rib 629 that can extend from the elongate portion 626 into the suction volume 638 and that is configured to selectively engage a portion of the target tissue when disposed in the suction volume 638, as described in detail above.

The elongate portion 626 defines a lumen 627 that is configured to receive, for example, a sheath (e.g., the sheath 160 and/or 260), an implant, a pharmaceutical, and/or any suitable portion of an insertion mechanism such as a catheter, a tube, a rod, an instrument, and/or the like. In this manner, the elongate portion 626 can allow an implant, pharmaceutical, etc. to be advanced through the suction volume 638 to be delivered to a desired bodily tissue that can be, for example, in a distal position relative to the nozzle 620. Moreover, the elongate member 626 includes a distal tip 628 that is at least partially disposed is a distal position relative to the rib 639 (see e.g., FIG. 17). In some embodiments, the distal tip 628 can be substantially similar to the distal tip 428 described above with reference to FIG. 12. Thus, the distal tip 628 can reduce and/or substantially eliminate damage to bodily tissue during insertion of the nozzle 620. Although not shown in FIGS. 15-47, the distal tip 628 can be transitioned from a first configuration (e.g., a closed configuration as shown in FIG. 17) and a second configuration (e.g., an open configuration in which the lumen 627 extends therethrough). Thus, a sheath, implant, pharmaceutical, and/or any other suitable portion of the insertion mechanism or the like can be passed through the lumen 627 to be delivered to a desired bodily tissue (e.g., the fundus 14 of the uterus 11 (FIG. 1)), as described above.

In use, at least a portion of the device can be inserted into a body cavity and manipulated to place the nozzle 620 in contact with a target tissue. For example, in some instances, the distal end portion 612 of the retractor 610 can be inserted into the vagina 15 of a patient and advanced to place fee nozzle 620 in contact with a portion of the cervix 12 (i.e., the target tissue). Once in contact with the target tissue, the vacuum source can be actuated and in turn, a suction force can be produced in the lumen 624 of the port 623 and the vacuum pathway 650. Thus, with the annular opening 635 placing the vacuum pathway 650 in fluid communication with the suction volume 638 at least a portion of the suction force is exerted on or within the suction volume 638 that is operable to draw a portion of the target tissue into the suction volume 638. Moreover, with the portion of the target tissue drawn into the suction volume 638, the elongate portion 626 can extend beyond a portion of the target tissue (e.g., the elongate portion 626 can extend through the cervical os (not shown) such that at least a portion of the distal tip 628 is positioned within the uterus (not shown).

As described above, the target tissue can be drawn into the suction volume 638 such that a surface of the rib 639 is placed in contact with a surface of the target tissue. More specifically, since the rib 639 extends from the inner surface 636 (as described above), the rib 639 can define a diameter that is smaller than a diameter of the remaining portions of inner surface 636. Thus, with the target tissue disposed in the suction volume 638 and selectively in contact with the inner surface 636, the rib 639 can deform a corresponding portion of the target tissue and as such, can place at least a portion of the proximal surface of the rib in contact with the target tissue. Accordingly, the contact between the proximal surface of the rib 639 and the target tissue can limit movement of the target tissue in a direction away from the connection portion 621 (i.e., the distal direction). Similarly, the rib 629 of the elongate portion 626 can be placed in contact with a portion of the target tissue to limit movement of the target tissue in the direction away from the connection portion 621. In this manner, the suction force exerted on a first portion of the target tissue via the vacuum pathway 650 and the annular opening 635, as well as the contact between the rib 639 and a first portion of the target tissue, and the rib 629 and a second portion of the target tissue can retain the target tissue within the engagement portion 630 (e.g., the suction volume 638). Moreover, the target tissue can be retained within the engagement portion 630 with a desired force sufficient to substantially prevent the target tissue from being withdrawn from the engagement portion 630 when the device is manipulated to exert a traction force on the target tissue. As such, the target tissue can be manipulated, moved, and/or otherwise reoriented to facilitate the insertion of, for example, an implant or the like. For example, in some instances, the nozzle 620 can retain a portion of the target tissue in the engagement portion 630 (e.g., the suction volume 638), while a traction force is applied thereto, thereby facilitating access (e.g., for a sheath or other delivery mechanism) through the cervical os 13 and into the uterus 11, as described in detail above.

In some embodiments, an amount of suction force exerted on the target tissue can be increased or decreased by changing the arrangement of the engagement portion 630. For example, in some embodiments, the size of the ribs 639 and/or 629 can be increased or decreased to increase or decrease, respectively, a contact surface between the portion of the target tissue and, for example, the proximal surface of the ribs 639 and/or 629, Accordingly, an increase in a size of the contact surface can, for example, result in an increase in a force configured to resist the distal movement of the target tissue relative to the engagement portion 630 (as described above) without a need, for example, to increase a suction force (e.g., an increase in a negative pressure differential produced by the vacuum source).

In a similar manner, an increase in a volume of the vacuum pathway 650 and/or an increase in a size (e.g., area) of the annular opening 635 can increase a force exerted on the target tissue to retain the vacuum nozzle 620 in contact with the target tissue at higher pull forces. Thus, by increasing the volume of the vacuum pathway 650 a suction force as result of a negative pressure differential produced by the vacuum source 690 can be reduced, while still retaining the target tissue within the engagement portion 630 during traction, as described in detail above with reference to the nozzle 420.

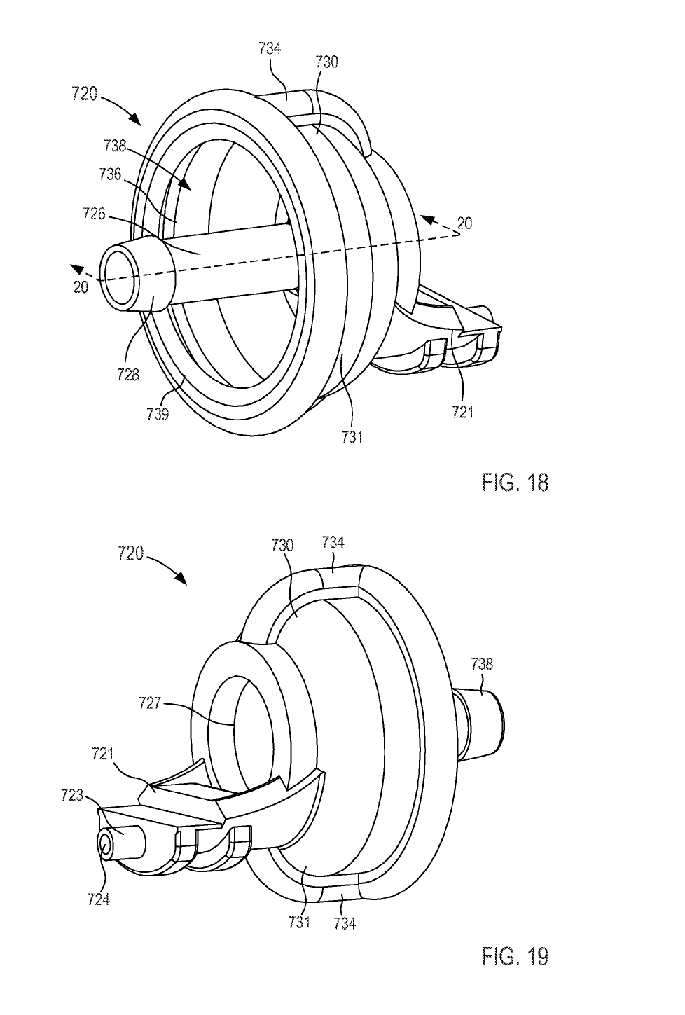

While the vacuum nozzles 320, 420, 520, and 620, are shown and described above as including and/or defining the vacuum pathways 350, 450, 550, and 650, respectively, at or in proximal portion of the nozzles 320, 420, 520, and 620, respectively, in other embodiments, a nozzle can form and/or can define a vacuum pathway at any suitable portion of the nozzle. For example, FIGS. 18-24 illustrate a vacuum nozzle 720 (also referred to herein as “nozzle” or “head”) according so another embodiment. The nozzle 720 can be used with any suitable device such as, for example, the retractor 410 with reference to FIGS. 8 and 9. The nozzle 720 includes a connection portion 721 and an engagement portion 730. Aspects of the vacuum nozzle 720 can be substantially similar in form and/or function as the vacuum nozzle 420 described above with reference to FIGS. 8-13. Thus, such aspects of the vacuum nozzle 720 that are similar to corresponding aspects of the vacuum nozzle 420 are not described in further detail herein.

The connection portion 721 of the nozzle 720 includes a vacuum port 723 (also referred to herein as “port”) and is configured to movably couple the nozzle 720 to the device (not shown). The port 723 of the connection portion 721 defines a lumen 724 in fluid communication with a vacuum source (not shown in FIGS. 18-24) and a portion of the engagement portion 730 (see e.g., FIG. 17). The vacuum source can be any suitable device, mechanism, assembly, etc. configured to produce a negative pressure differential once actuated, as described in detail above with reference to the nozzle 420 in FIGS. 8-13.

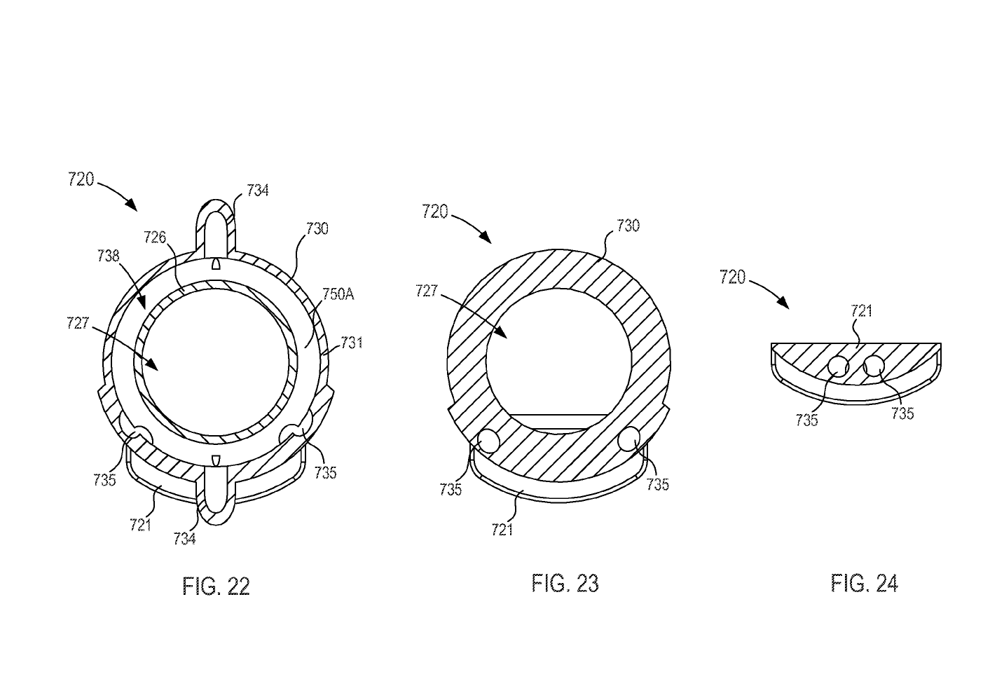

The engagement portion 730 of the nozzle 720 is coupled to the connection portion 721 and is configured to receive a portion of a target tissue (as described in further detail herein). The engagement portion 730 can be any suitable shape, size, and/or configuration, for example, in some embodiments, the engagement portion 730 can be substantially cylindrical including and/or otherwise being formed from a set of annular walls 731. As such, the annular walls 731 include an inner surface 736 that defines a suction volume 738 configured to be in fluid communication with the suction port 723. More specifically, the engagement portion 730 defines a pair of openings 735 that places the suction volume 738 in fluid communication with the lumen 724 of the port 723 (see e.g., FIGS. 22-24). In this manner, when the vacuum source is actuated, a negative pressure (e.g., a suction force) is produced within the suction volume 738 that can be operable in retaining a target tissue within at least a portion of the suction volume 738, as described in further detail herein.

The inner surface 736 includes and/or forms a rib 739 disposed at a distal end of the set of annular walls 731. The inner surface 736 also defines a first vacuum pathway 750A disposed at or adjacent to a proximal end portion of the set of annular walls 731 and a second vacuum pathway 750B disposed at or adjacent to a distal end portion of the set of annular walls 731, as described in further detail herein. The rib 739 can be, for example, a protrusion, a tab, a ridge, a rail, a flange, a ring, and/or like that extends from the inner surface 736 into the suction volume 738. In some embodiments, the rib 739 can be substantially similar to the rib 439 of the nozzle 420, as described in detail above with reference to FIG. 12. For example, the rib 739 can be configured to engage and/or deform a portion of the target tissue when disposed in the suction volume 738, which can be operable in retaining a target tissue within the suction volume 738, as described above with reference to the nozzle 420.

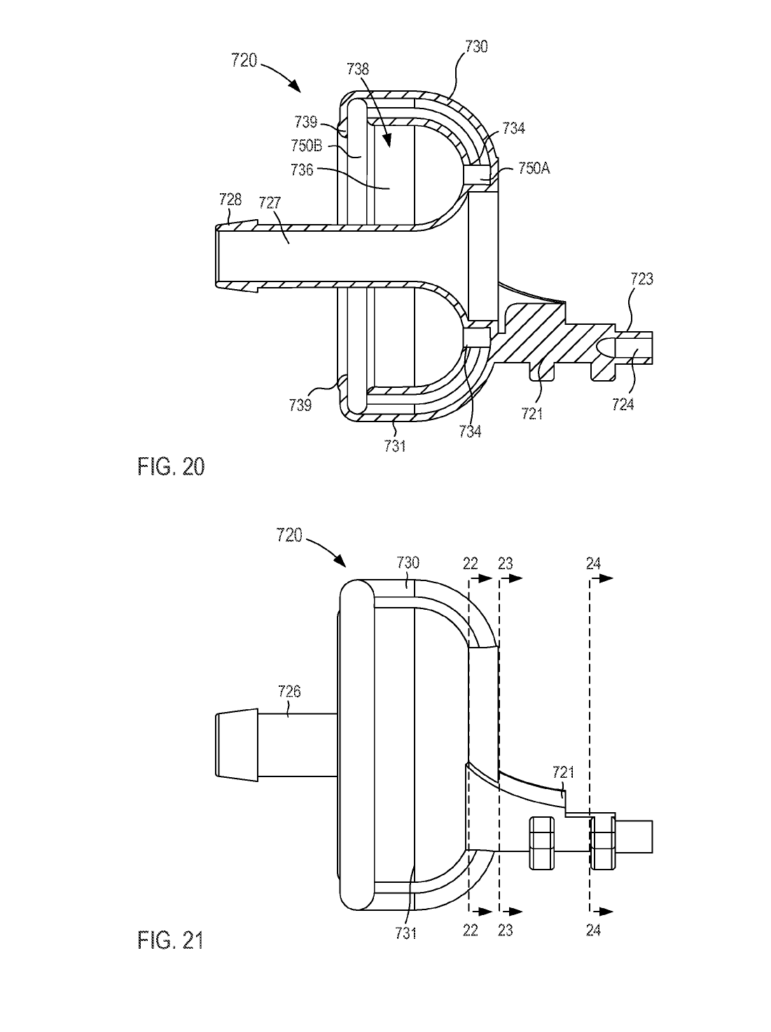

The first vacuum pathway 750A and the second vacuum pathway 750B can be any suitable configuration. For example as shown in FIGS. 18, 20, and 22, the first vacuum pathway 750A and the second vacuum pathway 750B can be, for example, grooves, channels, notches, and/or the like defined by the inner surface 731. More specifically, the first vacuum, pathway 750A is a substantially annular channel or the like defined by a proximal portion of the inner surface 736, as shown in FIG. 22. Similarly, the second vacuum pathway 750B is a substantially annular channel or the like defined by a distal portion of the inner surface 736. The vacuum pathways 750A and 750B can be any suitable shape, size, and/or configuration. Moreover, the engagement portion 730 defines a set of channels 734 that fluidically couple the first vacuum pathway 750A to the second vacuum pathway 750B, as shown in FIG. 20.

As shown in FIGS. 22-24, the first vacuum pathway 750A is in fluid communication with the lumen 724 defined by the port 723 via the openings 735. As such, a negative pressure produced by the vacuum source can result in a suction force within the first vacuum passway 750A. In this manner, the first vacuum pathway 750A substantially circumscribes a proximal surface of the engagement portion 730 and thus, defines a continuous volume that can circumscribe a portion of the target tissue (when disposed in the suction volume 738) and that can maintain substantially continuous communication with the port 723. As such, the suction force produced by the vacuum source can be substantially consistent within the vacuum pathway 750 and in turn, the suction force can be distributed with substantial uniformity about the portion to the target tissue. Furthermore, the channels 734 place the second vacuum pathway 750B in fluid communication with the first vacuum pathway 750A and thus, the target tissue can be disposed within the suction volume 738 and in contact with the inner surface 736 without having a portion that is spaced apart, as described above with reference to the nozzle 420.

The engagement portion 730 also includes an elongate portion 726 that extends from a proximal end portion of the engagement portion 730. The elongate portion 726 can be any suitable shape, size, or configuration. As shown in FIGS. 18, 20, and 21, the elongate portion 726 extends substantially though a center of the engagement portion 730. In other embodiments, an elongate portion can be offset from a center of an engagement portion. The elongate portion 726 defines a lumen 727 that is configured to receive, for example, a sheath (e.g., the sheath 160 and/or 260), an implant, a pharmaceutical, and/or any suitable portion of an insertion mechanism such as a catheter, a tube, a rod, an instrument, and/or the like. In this manner, the elongate portion 726 can allow an implant, pharmaceutical, etc. to be advanced through the suction volume 738 to be delivered to a desired bodily tissue that can be, for example, in a distal position relative to the nozzle 720. Thus, a sheath, implant, pharmaceutical, and/or any other suitable portion of the insertion mechanism or the like can be passed through the lumen 727 to be delivered to a desired bodily tissue (e.g., the fundus 14 of the uterus 11 (FIG. 1)), as described above. Although not shown in FIGS. 18-24, in some embodiments, the elongate portion 726 can include and/or can be coupled to a distal tip that can be substantially similar to those described above with reference to the elongate portions 426, 526, and/or 626. In this manner, the elongate portion 726 can be substantially similar in at least form and/or function to any of the elongate portions 426, 526, and/or 626 described above and therefore, is not described in further detail herein.

In use, at least a portion of the device can be inserted into a body cavity and manipulated to place the nozzle 720 in contact with a target tissue. For example, in some instances, the distal end portion 712 of the retractor 710 can be inserted into the vagina 15 of a patient and advanced to place the nozzle 720 in contact with a portion of the cervix 12 (i.e., the target tissue). Once in contact with the target tissue, the vacuum source can be actuated and in turn, a suction force can be produced in the lumen 724 of the port 723, the first vacuum pathway 750A, the channels 734, and the second vacuum pathway 750B. Thus, with the vacuum pathways 750A and 750B in fluid communication with the suction volume 738 at least a portion of the suction force is exerted on or within the suction volume 738 that is operable to draw a portion of the target tissue into the suction volume 738. Moreover, with the portion of the target tissue drawn into the suction volume 738, the elongate portion 726 can extend beyond a portion of the target tissue (e.g., the elongate portion 726 can extend through the cervical os (not shown) such that at least a portion of the distal tip 728 is positioned within the uterus (not shown).

As described above, the target tissue can be drawn into the suction volume 738 such that a surface of the rib 739 is placed in contact with a surface of the target tissue. More specifically, since the rib 739 extends from the inner surface 736 (as described above), the rib 739 can define a diameter that is smaller than a diameter of the remaining portions of inner surface 736. Thus, with the target tissue disposed in the suction volume 738 and selectively in contact with the inner surface 736, the rib 739 can deform a corresponding portion of the target tissue and as such, can place at least a portion of the proximal surface of the rib in contact with the target tissue. Accordingly, the contact between the proximal surface of the rib 739 and the target tissue can limit movement of the target tissue in a direction away from the connection portion 721 (i.e., the distal direction), as described in detail above. Moreover, with the second vacuum pathway 750B disposed in a proximal position relative to the rib 739, a portion of the suction force can draw a portion of the target tissue toward the inner surface 736 and as such, can increase an amount of the target tissue in contact with, for example, a proximal surface of rib 739. Thus, the portion of the suction force exerted on a first portion of the target tissue via the first vacuum pathway 750A, the portion of the suction force exerted on a second portion of the target tissue via the second vacuum pathway 750B, and the contact between the rib 739 and the second portion of the target tissue can retain the target tissue within the engagement portion 730 (e.g., the suction volume 738). Moreover, the target tissue can be retained within the engagement portion 730 with a desired force sufficient to substantially prevent the target tissue from being withdrawn from the engagement portion 730 when the device is manipulated to exert a traction force on the target tissue. As such, the target tissue can be manipulated, moved, and/or otherwise reoriented to facilitate the insertion of) for example, an implant or the like. For example, in some instances, the nozzle 720 can retain a portion of the target tissue its the engagement portion 730 (e.g., the suction volume 738), while a traction force is applied thereto, thereby facilitating access (e.g., for a sheath or other delivery mechanism) through the cervical os 13 and into the uterus 11, as described in detail above.

In some embodiments, an amount of suction force exerted on the target tissue can be increased or decreased by changing the arrangement of the engagement portion 730. For example, in some embodiments, the size of the rib 739 can be increased or decreased to increase or decrease, respectively, a contact surface between the portion of the target tissue and, for example, the proximal surface of the rib 739. Accordingly, an increase in a size of the contact surface can, for example, result in an increase in a force configured to resist the distal movement of the target tissue relative to the engagement portion 730 (as described above) without a need, for example, to increase a suction force (e.g., an increase in a negative pressure differential produced by the vacuum source). In a similar manner, an increase in a volume or area of the first vacuum pathway 750A and/or the second vacuum pathway 750B can increase a force exerted on the target tissue to retain the vacuum nozzle 720 in contact with the target tissue at higher pull forces. Thus, by increasing the volume or area of the vacuum pathways 750A and/or 750B a suction force as result of a negative pressure differential produced by the vacuum source 790 can be reduced, while still retaining the target tissue within the engagement portion 730 during traction, as described in detail above with reference to the nozzle 420.

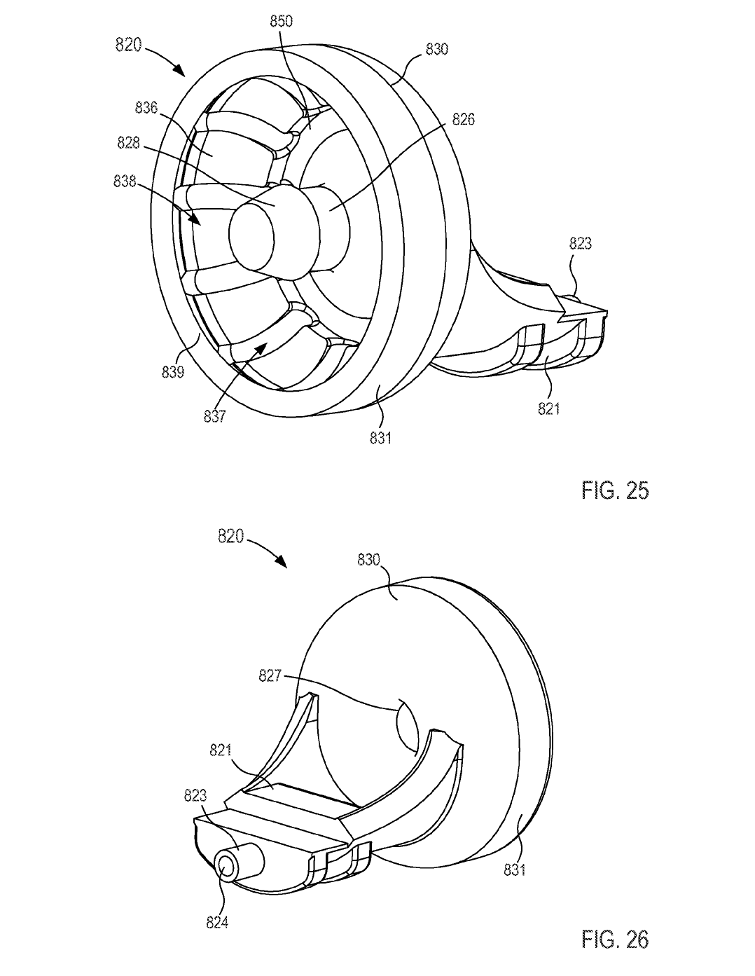

FIGS. 25-29 illustrate a vacuum nozzle 820 (also referred to herein as “nozzle”) according to another embodiment. The nozzle 820 can be used with any suitable device such as, for example, the retractor 410 with reference to FIGS. 8 and 9. The nozzle 820 includes a connection portion 821 and an engagement portion 830. Aspects of the vacuum nozzle 820 can be substantially similar in form and/or function as the vacuum nozzle 420 described above with reference to FIGS. 8-13. Thus, such aspects of the vacuum nozzle 820 that are similar to corresponding aspects of the vacuum nozzle 420 are not described in further detail herein.

The connection portion 821 of the nozzle 820 includes a vacuum port 823 (also referred to herein as “port” or “head”) and is configured to movably couple the nozzle 820 to the device (not shown). The port 823 of the connection portion 821 defines a lumen 824 in fluid communication with a vacuum source (not shown in FIGS. 25-29) and a portion of the engagement portion 830 (see e.g., FIGS. 28 and 29). The vacuum source can be any suitable device, mechanism, assembly, etc. configured to produce a negative pressure differential once actuated, as described in detail above with reference to the nozzle 420 in FIGS. 8-13.

The engagement portion 830 of the nozzle 820 is coupled to the connection portion 821 and is configured to receive a portion of a target tissue (as described in further detail herein). The engagement portion 830 cats be any suitable shape, size, and/or configuration. For example, in some embodiments, the engagement portion 830 can be substantially cylindrical including and/or otherwise being formed from a set of annular walls 831. As such, the annular walls 831 include an inner surface 836 that defines a suction volume 838 configured to be in fluid communication with the suction port 823. More specifically, the engagement portion 830 defines a pair of openings 835 that places the suction volume 838 in fluid communication with the lumen 824 of the port 823 (see e.g., FIGS. 28 and 29), as described in detail above.

The inner surface 836 includes and/or forms a rib 839 disposed at a distal end of the set of annular walls 831. The inner surface 836 also defines vacuum pathway 850 and a set of groove 837 or channels that extend from the vacuum pathway 850 toward a distal portion of the inner surface 836, as shown in FIG. 25. The rib 839 can be, for example, a protrusion, a tab, a ridge, a rail, a flange, a ring, and/or like that extends from the inner surface 836 into the suction volume 838. In some embodiments, the rib 839 can be substantially similar to the rib 439 of the nozzle 420, as described in detail above with reference so FIG. 12. For example, the rib 839 can be configured to engage and/or deform a portion of the target tissue when disposed in the suction volume 838, which can be operable in retaining a target tissue within the suction volume 838, as described above with reference to the nozzle 420.

The vacuum pathway 850 can be any suitable configuration. For example, as shown in FIG. 24, a proximal portion of the inner surface 836 defines an annular channel or the recessed portion that forms and/or defines the vacuum pathway 850. Although not shown in FIGS. 25-29, the vacuum pathway 850 is in fluid communication with the lumen 824 defined by the port 823 via the opening 835. As such, a negative pressure produced by the vacuum source can result in a suction force within the vacuum pathway 850. In addition, the vacuum pathway 850 is in fluid communication with the suction volume 838 (see e.g., FIG. 25 and 28). More specifically, the vacuum pathway 850 substantially circumscribes a proximal portion of the inner interface 836 and thus, defines a continuous volume that can circumscribe a portion of the target tissue (when disposed in the suction volume 838) and that can maintain substantially continuous communication with the port 823. As such, the suction force produced by the vacuum source can be substantially consistent within the vacuum pathway 850 and in turn, the suction force can be distributed with substantial uniformity about the portion to the target tissue. Furthermore, with the set of grooves 837 in fluid communication with the vacuum pathway 850, a circumferential surface of a portion of the target tissue can be exposed to a portion of the suction force (produced by the vacuum source) via the set of grooves 837.

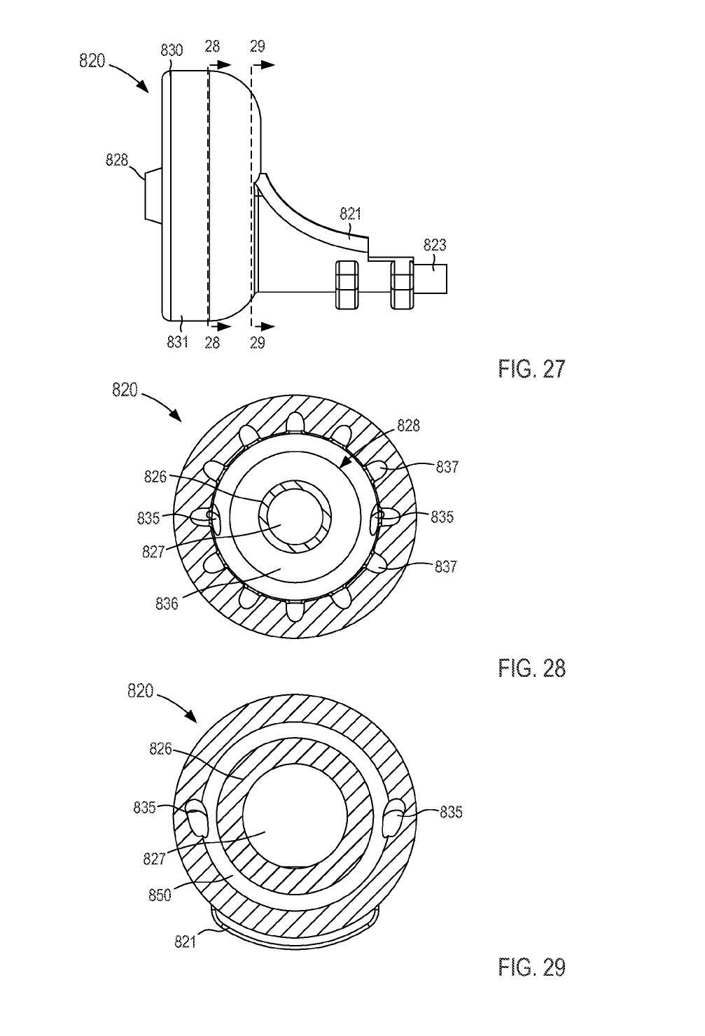

The engagement portion 830 also includes an elongate portion 826 that extends from a proximal end portion of the engagement portion 830. The elongate portion 826 can be any suitable shape, size, or configuration. As shown in FIGS. 25 and 27, the elongate portion 826 extends substantially though a center of the engagement portion 830. In other embodiments, an elongate portion can be offset from a center of an engagement portion. The elongate portion 826 defines a lumen 827 that is configured to receive, for example, a sheath (e.g., the sheath 160 and/or 260), an implant, a pharmaceutical, and/or any suitable portion of an insertion mechanism such as a catheter, a tube, a rod, an instrument, and/or the like. In this manner, the elongate portion 826 can allow an implant, pharmaceutical, etc. to be advanced through the suction volume 838 to be delivered to a desired bodily tissue that can be, for example, in a distal position relative to the nozzle 820. Thus, a sheath, implant, pharmaceutical, and/or any other suitable portion of the insertion mechanism or the like can be passed through the lumen 827 to be delivered to a desired bodily tissue (e.g., the fundus 14 of the uterus 11 (FIG. 1)), as described above. Although not shown in FIGS. 25-29, in some embodiments, the elongate portion 826 can include and/or can be coupled to a distal tip that can be substantially similar so those described above with reference to the elongate portions 426, 526, 626, and/or 726. In this manner, the elongate portion 826 can be substantially similar in at least form and/or function to any of the elongate portions 426, 526, 626, and/or 726 described above and therefore, is not described in further detail herein.

In use, at least a portion of the device can be inserted into a body cavity and manipulated to place the nozzle 820 its contact with a target tissue. For example, in some instances, the distal end portion 812 of the retractor 810 can be inserted into the vagina 15 of a patient and advanced to place the nozzle 820 in contact with a portion of the cervix 12 (i.e., the target tissue). Once in contact with the target tissue, the vacuum source can be actuated and in turn, a suction force can be produced in the lumen 824 of the port 823, the vacuum pathway 850, and the set of grooves 837. Thus, at least a portion of the suction force is exerted on or within the suction volume 838 that is operable to draw a portion of the target tissue into the suction volume 838. Moreover, with the portion of the target tissue drawn into the suction volume 838, the elongate portion 826 can extend beyond a portion of the target tissue (e.g., the elongate portion 826 can extend through the cervical os (not shown) such that at least a portion of the distal sip 828 is positioned within the uterus (not shown).

As described above, the target tissue can be drawn into the suction volume 838 such that a surface of the rib 839 is placed in contact with a surface of the target tissue. More specifically, since the rib 839 extends from the inner surface 836 (as described above), the rib 839 can define a diameter that is smaller than a diameter of the remaining portions of inner surface 836. Thus, with the target tissue disposed its the suction volume 838 and selectively in contact with the inner surface 836, the rib 839 can deform a corresponding portion of the target tissue and as such, can place at least a portion of the proximal surface of the rib in contact with the target tissue. Accordingly, the contact between the proximal surface of the rib 839 and the target tissue can limit movement of the target tissue in a direction away from the connection portion 821 (i.e., the distal direction), as described in detail above. Moreover, with the grooves 837 defined, for example, in an array about the target tissue, a portion of the suction force can draw a portion of the target tissue toward the inner surface 836 and as such, can increase an amount of the target tissue in contact with, for example, a proximal surface of rib 839. Thus, the portion of the suction force exerted on the target tissue can be retained within the engagement portion 830 with a desired force sufficient to substantially prevent the target tissue from being withdrawn from the engagement portion 830 when the device is manipulated to exert a traction force on the target tissue. As such, the target tissue can be manipulated, moved, and/or otherwise reoriented to facilitate the insertion of, for example, an implant or the like, as described in detail above.

In some embodiments, an amount of suction force exerted on the target tissue can be increased or decreased by changing the arrangement of the engagement portion 830. For example, in some embodiments, the size of the rib 839 can be increased or decreased to increase or decrease, respectively, a contact surface between the portion of the target tissue and, for example, the proximal surface of the rib 839. Accordingly, an increase in a size of the contact surface can, for example, result in an increase in a force configured to resist the distal movement of the target tissue relative to the engagement portion 830 (as described above) without a need, for example, to increase a suction force (e.g., an increase in a negative pressure differential produced by the vacuum source). In a similar manner, an increase in a volume of the vacuum pathway 850 and/or a volume or arrangement of the grooves 837 can increase a force exerted on the target tissue to retain the vacuum nozzle 820 in contact with the target tissue at higher pull forces, as described in detail above.

Although the grooves 837 are shown in FIGS. 25-29 as extending in a substantially linear path from the vacuum pathway 850 toward the rib 839, in other embodiments, an inner surface can define a set of grooves in any sui table configuration and/or orientation. For example, in some embodiments, the grooves 837 can be in a spiraled orientation, a lateral orientation, and/or any suitable pattern.

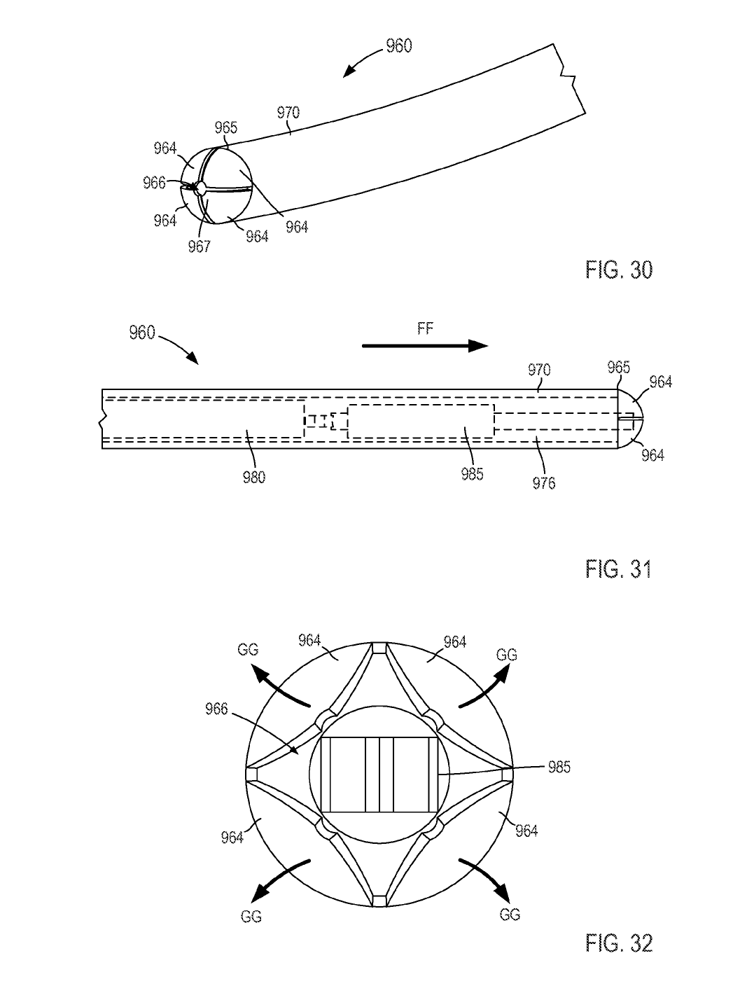

Any of the nozzles or heads 320, 420, 520, 620, 720, and/or 820 can be used, for example, with any suitable device configured to deliver an implant, a pharmaceutical, and/or foe like to a target location within the body. For example, the nozzles or heads 320, 420, 520, 620, 720, and/or 820 can be used with any suitable insertion device and/or tissue engagement device that can include and/or otherwise employ a sheath or the like such as the sheath 160 and/or 260 described above. Moreover, FIGS. 30-32 are illustrations of a portion of a sheath 960 according to an embodiment. In some embodiments, the sheath 960 can be used, for example, in conjunction with any of the nozzles 320, 420, 520, 620, 720, and/or 820 described above. In other embodiments, the sheath 960 can be used, for example, with an insertion device and/or tissue engagement independent from the nozzles 320, 420, 520, 620, 720, and/or 820.

In some embodiments, the sheath 960 can be used, for example, to deliver and/or place an IUD in contact with the fundus 14 of or otherwise within the uterus 10 (see e.g., FIG. 1). The sheath 960 can be formed from any suitable material or combination of materials such as, for example, those described above. More specifically, the sheath 960 can be formed or constructed from a substantially flexible material (e.g., a relatively high durometer rubber, siliconized rubber, polypropylene, polyethylene, and/or the like) that can allow for bending, twisting, opening, and/or otherwise reconfiguring of at least a portion of the sheath 960. For example, the sheath 960 can be sufficiently flexible to be advanced along a tortuous path defined be a portion of the body, yet can be sufficiently stiff to resist kinking, buckling, collapsing, and/or plastically deforming.

As shown, the sheath 960 includes at least an exit portion 970 and defines a lumen 976 (FIG. 31). The lumen 976 movably receives a portion of an insertion member 980 that is configured to be placed in contact with the IUD 985, For example, the IUD 985 can be loaded into the sheath 960 to be movably disposed in the lumen 976 and similarly, the insertion member 980 can be inserted into the lumen 976 to be placed in contact with the IUD 985, as described in further detail herein. As shown in FIGS. 30-32, in this embodiment, the exit portion 970 is a distal end portion of the sheath 960 and the lumen 976 can be configured to extend therethrough.

The exit portion 970 of the sheath 960 includes or is otherwise coupled to a set of dilation members 964. For example, in this embodiment, the set of dilation members 964 can be monolithically formed with the sheath 960 (i.e., the exit portion 970 of the sheath 960). More specifically, the dilation members 964 can be included in or otherwise coupled to the exit portion 970 in such a manner as to allow the dilation members 964 to be moved and/or transitioned between a first configuration (FIGS. 30 and 31) and a second configuration (FIG. 32). For example, in some embodiments, each dilation member 964 can be movably coupled to the exit portion 970 of the sheath 960 via a hinge 965 or the like. Expanding further, the hinge 965 can be, for example, a living hinge defined along and/or around a circumference of the exit portion 970 and configured to deform in a predetermined manner to move the dilation members 964 relative to the sheath 960. For example, the exit portion 970 can include a surface (e.g., an outer surface, an inner surface, or both) that is thinned, stretched, perforated, grooved, notched, scored, and/or otherwise weakened (e.g., made more flexible) to define the hinges 965 (i.e., a living hinge). Thus, each dilation member 964 can be configured to pivot and/or rotate about an axis (not shown) defined by the corresponding hinge 965 to be placed in the second configuration, as shown in FIG. 31.

Each dilation member 964 can be any suitable shape, size, and/or configuration. Similarly, the set of dilation members 964 can be any suitable arrangement. For example, while the set of dilation members 964 is shown in FIGS. 30-32 as including four dilation members 964, in other embodiments, the set of dilation members 964 can including a single dilation member, two dilation members, three dilation members, five dilation members, or more. Moreover, the set of dilation members 964 forms a dilation surface 967 that is substantially dome-shaped or the like, in this manner, the dilation surface 967 can be arranged in such a manner as to reduce and/or substantially eliminate sharp corners and/or angles that can, in some instances, result in the sheath 960 scraping and/or becoming caught on a surface of the bodily tissue. With the dilation members 964 forming, at the least, rounded corners, the exit portion 970 and/or the dilation members 964 can be advanced along a surface of the bodily tissue substantially without becoming caught thereon.

The set of dilation members 964 collectively form the dilation surface 967 that can define an opening 966. More specifically, when the dilation members 964 are in the first configuration, the opening 966 can have a first size and/or diameter, in some instances, the opening 966 can be relatively small to at least partially isolate the lumen 976 from a volume outside of the sheath 960, while allowing the dilation members 964 to move, flex, and/or bend during insertion. In some embodiments, the arrangement of the dilation members 964, the dilation surface 967 and/or the exit portion 970 can allow the IUD 985 to be loaded therethrough. That is so say, in some instances, the IUD 985 can be inserted through the dilation members 964 to be disposed in the lumen 976 prior so the sheath 960 being inserted into the body. The sheath 960 can be transitioned from its first configuration to its second configuration by moving the insertion member 980 in the distal direction, as indicated by the arrow FF in FIG. 31. More particularly, with the IUD 985 in contact with the insertion member 980, the distal movement insertion member 980 moves the IUD 985 within the lumen 976 in the FF direction. As such, the IUD 985 can contact the set of dilation members 964 so transition the each dilation member 964 from the first configuration so the second configuration, as indicated by the arrows GG in FIG. 32. Thus, the opening 966 defined by the dilation surface 967 can be dilated and/or otherwise enlarged to allow the IUD 985 to be conveyed from the lumen 976. Thus, the insertion member 980 can move the IUD 985 in the distal direction (e.g., the FF direction) to place the IUD 985 (e.g., an IUD) at a target location within the body (e.g., the fundus 14 of the uterus 11).

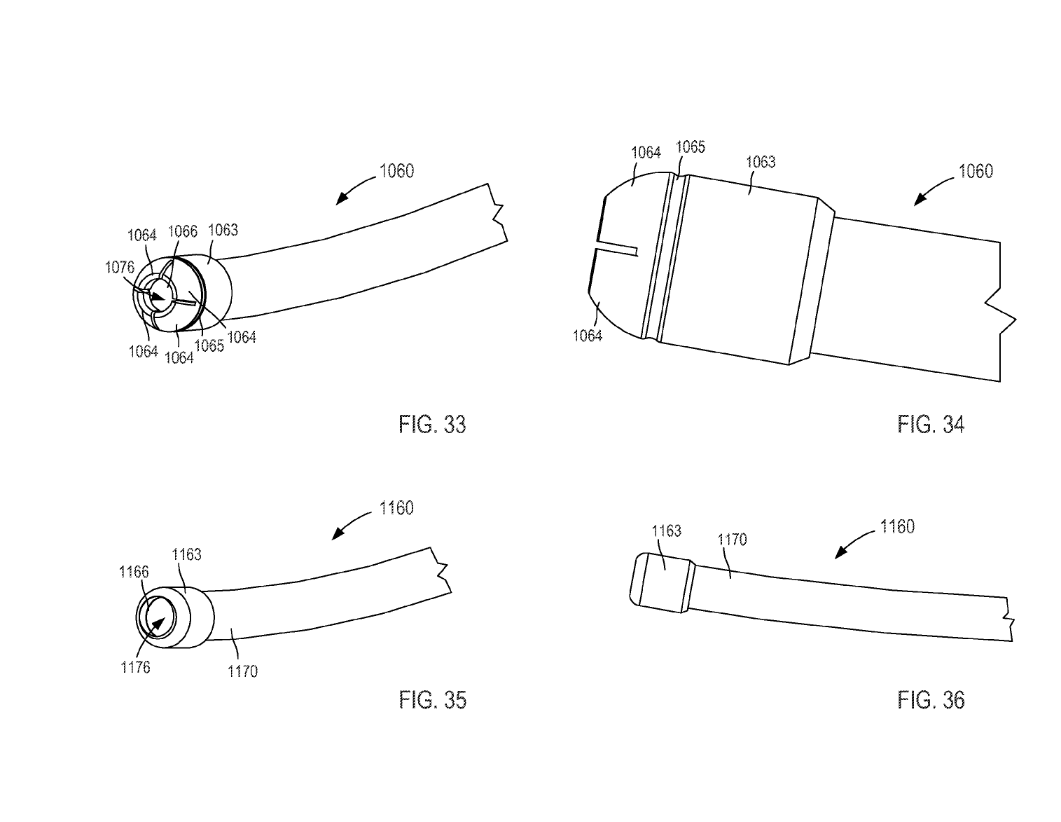

FIGS. 33 and 34 are illustrations of a portion of a sheath 1060 according to another embodiment. In some embodiments, the sheath 1060 can be used, for example, in conjunction with any of the nozzles 320, 420, 520, 620, 720, and/or 820 described above. In other embodiments, the sheath 1060 can be used, for example, with an insertion device and/or tissue engagement device independent from the nozzles 320, 420, 520, 620, 720, and/or 820. In some embodiments, the sheath 1060 can be used, for example, to deliver and/or place an IUD in contact with the fundus 14 of or otherwise within the uterus 10 (see e.g., FIG. 1). As described above, the sheath 1060 can be formed from any suitable material or combination of materials such as, for example, those described above. More specifically, the sheath 1060 can be formed or constructed from a substantially flexible material that can allow for bending, twisting, opening, and/or otherwise reconfiguring of at least a portion of the sheath 1060. For example, the sheath 1060 can be sufficiently flexible to be advanced along a tortuous path defined be a portion of the body, yet can be sufficiently stiff to resist kinking, buckling, collapsing, and/or plastically deforming.

As shown, the sheath 1060 includes at least an exit portion 1070 and defines a lumen 1076 (FIG. 33). The lumen 1076 movably receives a portion of an insertion member (not shown in FIGS. 33 and 34) that is configured to be placed its contact with the IUD (not shown in FIGS. 33 and 34), as described above with reference to the sheath 960. As shown in FIGS. 33 and 34, in this embodiment, the exit portion 1070 is a distal end portion of the sheath 1060 and the lumen 1076 can be configured to extend therethrough. The exit portion 1070 of the sheath 1060 includes or is otherwise coupled to a set of dilation members 1064. For example, in this embodiment, the set of dilation members 1064 are included in, for example, a distal tip 1063 or the like that can be coupled to the exit portion 1070 of the sheath 1060. For example, the distal tip 1063 can be an over-mold or the like. In other embodiments, such a distal tip can be formed from a material that is co-extruded with the sheath 1060. In this manner, the distal tip 1063 and the set of dilation members 1064 included therein can be formed from a substantially flexible material with a relatively low hardness (e.g., different from the material forming the sheath 1060). For example, the distal tip 1063 and/or the dilation members 1064 can be formed from a relatively low durometer rubber, silicone, siliconized rubber, and/or the like having a durometer that is less than a durometer of the sheath 1060. In some embodiments, the distal tip 1063 can be formed from a substantially fluid-impermeable foam such as foam rubber or the like. In this manner, the relatively low durometer of the distal tip 1063 can, for example, limit and/or substantially prevent damage to bodily tissue as the sheath 1060 is inserted into the body.

As shown in FIGS. 33 and 34, each dilation member 1064 can be movably coupled to a portion of the distal tip 1063 via a hinge 1065 or the like. Each hinge 1065 can be, for example, a living hinge or the like that can be configured to deform in a predetermined manner to move the corresponding dilation member 1064 relative to the sheath 1060. More specifically, dilation members 1060 can be monolithically formed with the distal tip 1063, which in turn, can include a surface that is thinned, stretched, or otherwise weakened (e.g., made more flexible) to define the hinges 1065 (e.g., a living hinge), as described above. Each dilation member 1064 can be any suitable shape, size, and/or configuration. Similarly, the set of dilation members 1064 can be any suitable arrangement. For example, while the set of dilation members 1064 is shown in FIG. 33 as including four dilation members 1064, in other embodiments, the set of dilation members 1064 can including a single dilation member, two dilation members, three dilation members, five dilation members, or more. Moreover, the set of dilation members 1064 forms a dilation surface 1067 that is substantially dome-shaped or the like and that defines an opening 1066. More specifically, when the dilation members 1064 are in the first configuration, the opening 1066 can have a first size and/or diameter. In some instances, the opening 1066 can be relatively small to at least partially isolate the lumen 1076 from a volume outside of the sheath 1060, while allowing the dilation members 1064 to move, flex, and/or bend during insertion. In addition, the dilation surface 1067 can be arranged in such a manner as to reduce and/or substantially eliminate sharp corners and/or angles that can, in some instances, result in the sheath 1060 scraping and/or becoming caught on a surface of the bodily tissue. With the dilation members 1064 forming, at the least, rounded corners, the distal tip 1063 and/or the dilation members 1064 can be advanced along a surface of the bodily tissue substantially without becoming caught thereon.

In some embodiments, the arrangement of the dilation members 1064, the dilation surface 1067 and/or the distal tip 1063 can allow the IUD to be loaded therethrough. That is to say, in some instances, the IUD can be inserted through the dilation members 1064 (i.e., the opening 1066) to be disposed in the lumen 1076 prior to the sheath 1060 being inserted into the body. With the IUD loaded in the lumen 1076, the sheath 1060 can be transitioned from a first configuration to a second configuration (not shown) by moving the insertion member 1080 in the distal direction. More particularly, the insertion member (not shown) disposed within the lumen 1076 can moves the IUD within the lumen 1076 to place the IUD in contact with the set of dilation members 1064, thereby transitioning each dilation member 1064 from the first configuration to the second configuration. Thus, the opening 1066 defined by the dilation surface 1067 can be dilated and/or otherwise enlarged to allow the IUD to be conveyed from the lumen 1076. Thus, the insertion member 1080 can move the IUD in the distal direction to place the IUD at a target location within the body (e.g., the fundus 14 of the uterus 11).

Although the distal tip 1063 is shown and described above as including the set of dilation members 1064, in other embodiment, a sheath can be coupled to a distal tip that does not include a set of dilation members. For example, FIGS. 35 and 36 are illustrations of a portion of a sheath 1160 according to an embodiment. In some embodiments, the sheath 1160 can be used, for example, in conjunction with any of the nozzles 320, 420, 520, 620, 720, and/or 820 described above. In other embodiments, the sheath 1160 can be used, for example, with an insertion device and/or tissue engagement device independent from the nozzles 320, 420, 520, 620, 720, and/or 820. In some embodiments, the sheath 1160 can be used, for example, to deliver and/or place an IUD in contact with the fundus 14 of or otherwise within the uterus 10 (see e.g., FIG. 1). The sheath 1160 can be formed from any suitable material or combination of materials such as, for example, those described above. More specifically, the sheath 1160 can be formed or constructed from a substantially flexible material (e.g., a relatively high durometer rubber, siliconized rubber, polypropylene, polyethylene, and/or the like) that can allow for bending, twisting, opening, and/or otherwise reconfiguring of at least a portion of the sheath 1160, as described in detail above.

As shown, the sheath 1160 includes at least an exit portion 1170 and defines a lumen 1176 (FIG. 35). The lumen 1176 movably receives a portion of an insertion member (not shown in FIGS. 35 and 36) that is configured to be placed in contact with the IUD (not shown in FIGS. 35 and 36), as described above with reference to the sheath 960. In this embodiment, the exit portion 1170 is a distal end portion of the sheath 1160 and the lumen 1176 can be configured to extend therethrough. The exit portion 1170 of the sheath 1160 includes or is otherwise coupled to a distal tip 1163. For example, the distal tip 1163 can be an over-mold or the like. In other embodiments, the distal tip 1163 can be formed from a material that is co-extruded with the sheath 1160. In this manner, the distal tip 1163 can be formed from a substantially flexible material with a relatively low hardness (e.g., different from the material forming the sheath 1160). For example, the distal tip 1163 can be formed from a relatively low durometer rubber, silicone, siliconized rubber, and/or the like having a durometer that is less than a durometer of the sheath 1160. In some embodiments, the distal tip 1163 can be formed from a substantially fluid-impermeable foam such as foam rubber or the like. In this manner, the relatively low durometer of the distal tip 1163 can, for example, limit and/or substantially prevent damage to bodily tissue as the sheath 1160 is inserted into the body. The distal tip 1163 defines an opening 1166 through which the IUD can be advanced. Moreover, the distal tip 1163 can be arranged in such a manner as to reduce and/or substantially eliminate sharp corners and/or angles that can, in some instances, result in the sheath 1160 scraping and/or becoming caught on a surface of the bodily tissue. Thus, the sheath 1160 can be inserted into a portion of the body to place the distal tip 1163 at a desired location therein. Once the distal tip 1163 is disposed in the desired location, the insertion member 1180 can move the IUD in the distal direction to covey the IUD from the lumen 1174 to a target location within the body (e.g., the fundus 14 of the uterus 11).

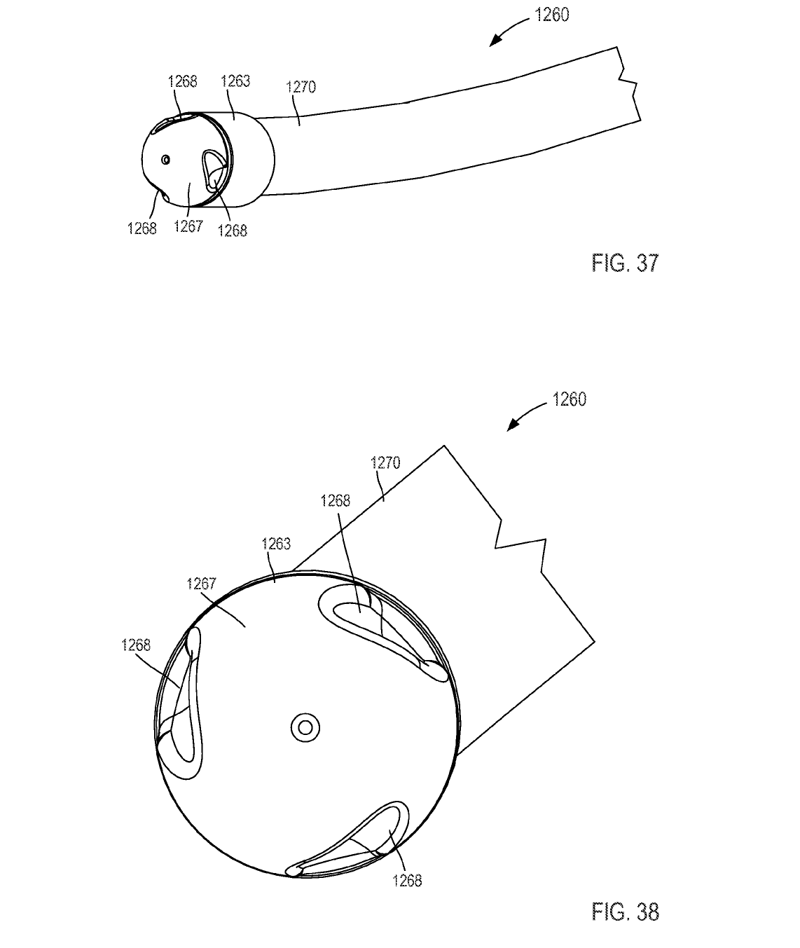

Although the exit portions, distal tips, and/or dilation members are shown and described above with reference to FIGS. 30-36 as forming a rounded and/or domed-shape dilation or distal surface, in other embodiments, a distal tip can form any suitable shape. For example, FIGS. 37 and 38 are illustrations of a portion of a sheath 1260 according to another embodiment. In some embodiments, the sheath 1260 can be used, for example, in conjunction with any of the nozzles 320, 420, 520, 620, 720, and/or 820 described above. In other embodiments, the sheath 1260 can be used, for example, with an insertion device and/or tissue engagement device independent from the nozzles 320, 420, 520, 620, 720, and/or 820. In some embodiments, the sheath 1260 can be used, for example, to deliver and/or place an IUD in contact with the fundus 14 of or otherwise within the uterus 10 (see e.g., FIG. 1). The sheath 1260 can be formed from any suitable material or combination of materials that can allow for bending, twisting, opening, and/or otherwise reconfiguring of at least a portion of the sheath 1260 such as, for example, those described above.

As shown, the sheath 1260 includes at least an exit portion 1270. Although not shown in FIGS. 37 and 38, the sheath 1260 can define a lumen configured to movably receive a portion of an insertion member (not shown in FIGS. 37 and 38) and the IUD (not shown in FIGS. 37 and 38), as described above with reference to the sheath 960. In this embodiment, the exit portion 1270 is a distal end portion of the sheath 1260. The exit portion 1270 of the sheath 1260 includes or is otherwise coupled to a distal tip 1263. For example, the distal tip 1263 can be an over-mold or the like. In other embodiments, the distal tip 1263 can be formed from a material that is co-extruded with the sheath 1260. In this manner, the distal tip 1263 can be formed from a substantially flexible material with a relatively low hardness (e.g., different from the material forming the sheath 1260). For example, the distal tip 1263 can be formed from a relatively low durometer rubber, silicone, siliconized rubber, and/or the like having a durometer that is less than a durometer of the sheath 1260, as described in detail above. In this manner, the relatively low durometer of the distal tip 1263 can, for example, limit and/or substantially prevent damage to bodily tissue as the sheath 1260 is inserted into foe body.

The distal tip 1263 includes a distal surface 1267 that can form, for example, a dome-shape or the like. That is to say, the distal surface 1267 can extend in a curvilinear path from a proximal position toward a distal position. In this manner, the rounded and/or domed shape of the distal surface 1267 can reduce and/or substantially eliminate sharp corners and/or angles that can, in some instances, result in the sheath 1260 scraping and/or becoming caught on a surface of the bodily tissue. Moreover, as shown in FIGS. 37 and 38, the distal surface 1267 defines a set of dimples 1268 which can be disposed, for example, at or adjacent to a base of the distal surface 1267 (e.g., at or adjacent to the proximal position of the distal surface 1267). Each dimple 1268 can be any suitable shape or size. For example, as shown in FIGS. 37 and 38, the dimples 1268 can be and/or can form indented portions of the distal surface 1267 with a substantially spiraled or otherwise curved orientation relative to the distal surface 1267. As shown, in this embodiment, the set of dimples 1268 includes, for example, three dimples equally spaced around a circumference of the distal surface 1267. In other embodiments, a distal surface can include any number of dimples, which can be uniformly or non-uniformly arranged around the distal surface. As such, the dimples can, for example, facilitate the insertion of the sheath 1260 through a bodily lumen or the like. For example, in some embodiments, the set of dimples 1268 can facilitate the navigation of at least the distal tip 1263 through a tortuous path and/or around partial occlusions such as, for example, fibroids or the like.

Although not shown in FIGS. 37 and 38, the distal surface 1267 can include an opening, a set of slits, a set of dilation members, and/or the like that can be configured to allow passage of, for example, the IUD therethrough. For example, the insertion member can move be moved within the lumen (not shown in FIGS. 37 and 38) to convey the IUD from the lumen to a target location within the body (e.g., the fundus 14 of the uterus 12). In some instances, the sheath 1260 and/or the distal tip 1263 can be used in conjunction with any suitable medical device during any suitable medical procedure such as any of those described herein.

In some embodiments, any of the nozzles 320, 420, 520, 620, 720, and/or 820 can be disconnected from a housing of a retractor or the like to be used as a separate device. Thus, in some embodiments, the nozzle can function substantially independently to perform functions similar to those performed by the cervical tenaculum in other intrauterine procedures, including, but not limited to, artificial insemination (intrauterine semination), colcoscopy, dilation and curettage, manual vacuum aspiration, electric vacuum aspiration, endometrial biopsy, dilatation and evacuation, insertion of various contraceptive devices, uterine fibroid removal and certain abortion procedures. This second embodiment includes the suction mechanism, including but not limited to a vacuum creating mechanism and the ports at the distal end of the device to create suction with the tissue with which it comes in contact. The suction will enable a user of the device to pull traction on the tissue up to certain level of force.

In some embodiments, any of the nozzles 320, 420, 520, 620, 720, and/or 820 can be moved in a direction via a spring action (e.g., a spring, spring fingers, leaf spring, preloaded member, etc.) or other biasing action. In such embodiments, the spring action can facilitate the alignment and/or positioning of the port to the cervix or the body part to which the port is attached. In some embodiments, a port of a nozzle can be coupled to a flexible tubing configured to fluidically couple the port to vacuum source and the flexible tubing can be configured to at least partially act as a biasing member. In some embodiments, a spring can be included in the retractor and coupled to the port and/or vice versa.

While various embodiments have been described above, it should be understood that they have been presented by way of example only, and not limitation. Where schematics and/or embodiments described above indicate certain components arranged in certain orientations or positions, the arrangement of components may be modified. While the embodiments have been particularly shown and described, it will be understood that various changes in form and details may be made. Although various embodiments have been described as having particular features and/or combinations of components, other embodiments are possible having a combination of any features and/or components from any of embodiments as discussed above. For example, an alternate embodiment can be created using any suitable portion or combination of parts of the embodiments described herein. For example, such an embodiment can form an improved tenaculum that provides temporary attachment to the cervix through vacuum/suction mechanism instead of currently used method of a sharp tongs-like mechanism.

By way of another example, although not shown, any of the nozzles 420, 520, 620, 720, and/or 820 can include a surface and/or portion that can be substantially transparent and/or that can be shaped like a lens or the like, as described above with reference to the nozzle 320 in FIG. 7.

Although some of the vacuum nozzles or suction heads are described herein as including a rib (e.g., the rib 439 of the head 420), in other embodiments, any of the vacuum nozzles or suction heads described herein can include any suitable number of ribs (or protrusion) in any suitable orientation. For example, in some embodiments, the suction nozzle 420 can include two or more ribs aligned circumferentially about the distal tip 428 (or dilation member). In this manner, the series of ribs can form a series of barbs or protrusions to assist in the retention of tissue within the vacuum nozzle,

Although the vacuum nozzle 420 is shown and described as including a single vacuum port 423, in other embodiments, any of the vacuum nozzles or suction heads described herein can include any suitable number of vacuum ports.

Where methods and/or events described above indicate certain events and/or procedures occurring in certain order, the ordering of certain events and/or procedures may be modified. Additionally, certain events and/or procedures may be performed concurrently in a parallel process when possible, as well as performed sequentially as described above.

1. An apparatus, comprising:
an insertion member having a distal end portion configured to be removably engaged with an implant; and
a sheath having an exit portion and defining a lumen, the exit portion of the sheath including a plurality of dilation members configured to be moved from a first configuration to a second configuration, the plurality of dilation members forming a dilation surface when the plurality of dilation members is in the first configuration, the plurality of dilation members defining an opening when the plurality of dilation members is in the second configuration, the sheath including a hinge configured to facilitate movement of the plurality of dilation members between the first configuration and the second configuration, the distal end portion of the insertion member configured to move within the lumen to convey the implant from within the lumen via the opening when the plurality of dilation members is in the second configuration.

2. The apparatus of claim 1, wherein the plurality of dilation members is included in a tip member, the tip member being removably coupled to the exit portion.

3. The apparatus of claim 1, wherein the sheath is formed from a first material having a first hardness, the apparatus further comprising:
a tip member including the plurality of dilation members and being coupled to the exit portion, the tip member being formed from a second material having a second hardness different than the first hardness.

4. The apparatus of claim 1, wherein:
the sheath includes a proximal portion having a first hardness; and
the exit portion has a second hardness different than the first hardness.

5. The apparatus of claim 1, wherein the sheath includes a tip member including the plurality of dilation members, the tip member being removably coupled to the exit portion, the tip member defining a groove to form at least a portion of the hinge.

6. The apparatus of claim 1, wherein the hinge is a living hinge.

7. The apparatus of claim 1, wherein the hinge includes a discontinuity defined by the exit portion.

8. The apparatus of claim 1, wherein the dilation surface is rounded to facilitate movement of the sheath within a lumen of a body.

9. The apparatus of claim 1, wherein the opening is a second opening, the plurality of dilation members collectively defining a first opening when the plurality of dilation members is in the first configuration, a diameter of the second opening greater than a diameter of the first opening.

10. An apparatus, comprising:
an insertion member having-a distal end portion configured to be removably -engaged with an implant to move the implant in a distal direction; and
a sheath helving a distal end portion, an exit portion and defining a lumen, the lumen configured to receive at least a portion of the insertion member and the implant, the distal end portion having a continuous dilation surface spaced apart from the lumen, the exit portion defining an opening in communication with the lumen, the exit portion including an exit surface defining an end portion of the lumen, the exit surface configured to contact a distal end portion of the implant when the insertion member moves the implant in the distal direction relative to the sheath to convey the implant from within the lumen via the opening.

11. The apparatus of claim 10, wherein the lumen defines a centerline the exit surface forming an acute angle with the centerline.

12. The apparatus of claim 10, wherein the lumen defines a centerline, the exit portion defining the opening offset from the centerline.

13. The apparatus of claim 10, wherein the dilation surface is dome-shaped.

14. The apparatus of claim 10, wherein the exit portion is associated with a first hardness and the distal end portion is associated with a second hardness different than the first hardness.

15. The apparatus of claim 10, wherein the insertion member is configured to be disposed within the lumen when the implant is conveyed though the opening.

16. An apparatus, comprising:
a connection portion configured to be pivotably coupled to a medical device, the connection portion including a vacuum port configured to be coupled to a vacuum source; and
an engagement portion coupled to the connection portion, the engagement portion including a rib and an inner surface, the inner surface defining at least, a portion of a vacuum pathway and at least a portion of a suction volume, the suction volume in fluid communication with the vacuum port via the vacuum pathway, the suction volume configured to receive a first portion of a target tissue when a portion of the rib is engaged with the target tissue and a vacuum is applied to the vacuum port, the inner surface configured such that the vacuum pathway provides continuous communication between the vacuum port and the suction volume when the first portion of the target tissue is within the suction volume, the rib configured to be in contact with a second portion of the target tissue when the first portion of target tissue is disposed in the suction volume to limit a movement of the target tissue out of the suction volume.

17. The apparatus of claim 16, wherein the rib has a first diameter and the inner surface has a second diameter greater than the first diameter.

18. The apparatus of claim 16, further comprising:
a dilation member, a distal surface of the dilation member disposed in a distal position relative to the rib of the engagement portion, the vacuum pathway circumscribing the dilation member.

19. The apparatus of claim 18, wherein the dilation member is configured to be disposed within a bodily opening of the target tissue when the first portion of the target tissue is within the suction volume.

20. The apparatus of claim 18, wherein the dilation member is configured to be transitioned from a first configuration to a second configuration when the target tissue is disposed in the suction volume, the distal surface of the dilation member defining an opening when in the second configuration, the delivery device configured to advance an implant through the opening.

21. The apparatus of claim 16, wherein the inner surface defines at least one groove configured to define a portion of the vacuum pathway.

22. The apparatus of claim 16, wherein the connection portion includes a window formed from a substantially transparent material configured to provide visual access to the suction volume.

23. The apparatus of claim 22, wherein the window is configured to magnify the first portion of the target, tissue.

24. The apparatus of claim 16, wherein the inner surface includes a curved portion defining at least a portion of the vacuum pathway, the curved portion defining a radius of curvature sized such that the curved portion surface is spaced apart from the target tissue when
the target tissue is disposed in the suction volume.

25. The apparatus of claim 16. further comprising:
a dilation member, a distal surface of the dilution member disposed in a distal position relative to the rib of the engagement portion, the dilation member having a tapered surface.

26. The apparatus of claim 25, wherein:
the dilation member is configured to be moved from a first configuration to a second configuration, the dilation member forming a dilation surface when the dilation member is in the first configuration, the dilation member defining an opening when the dilation member is in the second configuration.

27. The apparatus of claim 25, wherein the engagement portion is associated with a first hardness and the dilation member is associated with a second hardness different than the first hardness.
